(19) United States
(12) Patent Application Publication
(10) Pub. No.: US 2026/0283795 A1
(43) Pub. Date: Sep. 24, 2026

(54) TRANSCATHETER HEART VALVE DELIVERY SYSTEM SOLUTIONS

(71) Applicant: Edwards Lifesciences Corporation, Irvine, CA (US)

(72) Inventor: Paul Yen Tran, Anaheim, CA (US)

(21) Appl. No.: 19/688,428

(22) Filed: May 26, 2026

Related U.S. Application Data
(63) Continuation of application No. PCT/US2024/056863, filed on Nov. 21, 2024.
(60) Provisional application No. 63/603,068, filed on Nov. 27, 2023.

(51) Int. Cl. A61F 2/24; A61F 2/95; A61M 25/01

(52) U.S. Cl. CPC A61F 2/2436; A61F 2/9517; A61M 25/0136; A61M 25/0138; A61M 25/0147

(57) ABSTRACT

Systems, apparatuses, and methods disclosed herein are provided for transcatheter delivery. The system can include a flexible access sheath for advancement of an implant to an implantation site. The flexible access sheath can include an outer tube defining a first set of gaps and an inner tube defining a second set of gaps. The system can include a proximal control handle, to which the access sheath is attached. The control handle can include a pull wire and an actuator for rotating one of the inner tube and the outer tube relative to the other such that: (i) tensioning the pull wire while the first and second set of gaps are rotationally aligned bends the flexible access sheath toward the first and second set of gaps; and (ii) while the first and second set of gaps are rotationally misaligned bending of the flexible access sheath is inhibited.

### CROSS-REFERENCE TO PRIORITY APPLICATIONS

The present application is a continuation of International Application No. PCT/US2024/056863, filed Nov. 21, 2024, which designates the United States and was published in English by the International Bureau on Jun. 5, 2025 as WO2025117321, which claims priority to U.S. Provisional Application No. 63/603,068 filed Nov. 27, 2023, the entire content of which are hereby incorporated herein by reference.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Prostheses can be used to correct problems associated with impaired heart valves. For example, mechanical and tissue-based heart valve prostheses can be used to replace impaired native heart valves. More recently, substantial effort has been dedicated to developing replacement heart valves that can be delivered with less trauma to the patient than through open heart surgery. Replacement valves can be designed for percutaneous delivery in so-called transcatheter procedures.

Development of transcatheter heart valves and their delivery systems has proven to be particularly challenging. Delivering a replacement heart valve to the tricuspid valve, for instance, can be extremely challenging. Obtaining access to perform procedures in the heart or in other anatomical locations may require delivery of devices percutaneously through tortuous vasculature. To compound the difficulty, delivery systems for prosthetic heart valves have a practical maximum diameter to enable passage through the vasculature, which limits the number and type of delivery tools that can fit within the delivery sheath.

In light of the above, a need exists for more advanced transcatheter heart valve delivery systems.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, a system comprises a control handle (which can be the same as or similar to any of the control handles described anywhere herein). In some implementations, the system comprises a flexible sheath (which can be the same as or similar to any of the sheaths or catheters described anywhere herein) extending distally relative to the control handle and having a sheath lumen. In some implementations, a catheter (which can be the same as or similar to any of the catheters or delivery catheters described anywhere herein) having an outer diameter smaller than an inner diameter of the sheath lumen. In some implementations, the catheter has a catheter lumen extending therethrough.

In some implementations, the control handle has an actuator or a plurality of actuators. In some implementations, the catheter is linearly displaceable using an actuator of the control handle.

In some implementations, the system further comprises a stabilization system configured to hold stable the control handle. In some implementations, the stabilization system including a clamp assembly with movable jaws that secure a portion of the control handle. In some implementations, the portion comprises external threads and/or ribs.

In some implementations, the system further comprises a rotational adjustment bolt arranged to rotate about its axis while being held in a stationary position by the clamp assembly. In some implementations, the rotational adjustment bolt has threads and/or ribs that engage the external threads and/or ribs of the portion of the control handle. In some implementations, rotation of the rotational adjustment bolt rotates the control handle about an axis (e.g., a longitudinal axis along the length, etc.) of the control handle.

In some implementations, the clamp assembly includes a control knob for tightening and loosening the movable jaws in addition to the rotational adjustment bolt.

In some implementations, the clamp assembly has a toggle-like latch that closes the movable jaws and provides positional stability without creating a tight interference fit. In some implementations, the rotational adjustment bolt can rotate the control handle when the latch is closed.

In some implementations, the rotational adjustment bolt has a wingnut-type outer end for ease of grasping.

In some implementations, the flexible sheath comprises a distal segment extending from a first distal ring to a first proximal ring with a first flexible portion therebetween.

In some implementations, a proximal segment in series with the distal segment extends from a second distal ring to a second proximal ring with a second flexible portion therebetween.

In some implementations, a plurality of pull wires are coupled (e.g., directly coupled, indirectly coupled, etc.) to actuators of the control handle. In some implementations, the plurality of pull wires extend distally relative to the control handle.

In some implementations, a first set of pull wires of the plurality of pull wires extends through axial channels in the first proximal ring and second distal ring to be anchored to the first distal ring. In some implementations, a second set of pull wires of the plurality of pull wires is anchored to the second distal ring.

In some implementations, the plurality of pull wires are configured such that, when placed in tension via the actuators of the control handle, one or more pull wires of the plurality of pull wires can bend either or both of the distal segment and proximal segment in multiple planes.

In some implementations, the plurality of pull wires are configured such that, when placed in tension via the actuators of the control handle, one or more pull wires of the plurality of pull wires can bend either or both of the distal segment and proximal segment in at least 3 planes at different angles to each other. In some implementations, the plurality of pull wires are configured such that, when placed in tension via the actuators of the control handle, one or more pull wires of the plurality of pull wires can bend either or both of the distal segment and proximal segment in at least 4 planes at different angles to each other.

In some implementations, the plurality of pull wires are configured such that, when placed in tension via the actuators of the control handle, one or more pull wires of the plurality of pull wires can bend either or both of the distal segment and proximal segment in space relative to x, y, and z axes.

In some implementations, there are three wires in the first set of pull wires angularly spaced 120° apart. In some implementations, there are three wires in the second set of pull wires angularly spaced 120° apart and offset from the wires in the first set of pull wires by 60°.

In some implementations, there are three wires in the second set of pull wires angularly spaced 120° apart.

In some implementations, each pull wire of the plurality of pull wires can be independently actuated using actuators of the control handle.

In some implementations, the first proximal ring is conjoined with the second distal ring to form a single intermediate ring.

In some implementations, the proximal segment comprises a series of solid ribs that are flexibly connected together for bending and have larger gaps therebetween on a first circumferential side of the proximal segment than on a second circumferential side of the proximal segment. In some implementations, the distal segment comprises a series of solid ribs that are flexibly connected together for bending and have larger gaps therebetween on a first circumferential side of the distal segment than on a second circumferential side of the distal segment.

In some implementations, a first pull wire of the plurality of pull wires is anchored to a first circumferential side of the distal ring such that an actuator in the control handle can place the first pull wire in tension and bend the proximal segment in a first direction. In some implementations, a second pull wire of the plurality of pull wires is anchored to a second circumferential side of the distal ring, such that an actuator in the control handle can place the second pull wire in tension and bend the distal segment in a second direction different from the first direction.

In some implementations, the distal and proximal segments are adapted to bend in opposite directions in only one plane.

In some implementations, the proximal and distal segments comprise helically arranged coils with relatively tighter and looser coil spacing.

In some implementations, the flexible sheath includes a bendable segment (which can be configured similar to the proximal and distal segments above or configured similar to other bendable segments described elsewhere herein).

In some implementations, the bendable segment has an inner tube comprising a series of solid ribs that are flexibly connected for bending and having larger gaps therebetween on a first circumferential side of the inner tube than on a second circumferential side of the inner tube opposite the first circumferential side.

In some implementations, the bendable segment comprises an outer tube comprising a series of solid ribs that are flexibly connected for bending and having larger gaps therebetween on a first circumferential side of the outer tube than on a second circumferential side of the outer tube opposite the first circumferential side.

In some implementations, the control handle comprises an actuator that can be actuated to cause rotation of the inner tube relative to the outer tube, or vice versa.

In some implementations, the actuator (which can be actuated to cause rotation of the inner tube relative to the outer tube, or vice versa) can be actuated to align the larger gaps in the inner tube and the larger gaps in the outer tube to permit or enhance bending. In some implementations, the actuator can be actuated to misalign the larger gaps in the inner tube and the larger gaps in the outer tube to resist or impede bending.

In some implementations, the system further comprises a nose cone coupled to and projecting distally relative to a distal end of the delivery catheter. In some implementations, the nose cone is adapted to facilitate passage of the delivery catheter through vasculature of the subject.

In some implementations, the control handle comprises a plurality of annular members inside the control handle that are rotatable by external actuators on the control handle.

In some implementations, the control handle is detachable from the flexible sheath, catheter, and/or other components of the system.

In some implementations, the control handle is detachable from the catheter and/or other components of the system, and the control handle has a plurality of carriages, one for each of the catheter, one or more pull wires, and/or a nose cone.

In some implementations, the control handle is attachable to the catheter, the one or more pull wires, and/or the nose cone to place annular members of the control handle into engagement with the one or more carriages of the plurality of carriages so as to enable axial displacement of the catheter, the one or more pull wires, and/or the nose cone.

In some implementations, one or both of the flexible sheath and the catheter has a hub at a proximal end thereof. In some implementations, the hub has a pair of radially-outward lugs thereon. In some implementations, a distal end of the control handle has a hollow cylindrical housing defining two notches sized and positioned to receive the lugs, such that insertion and rotation of the lugs through the notches of the housing attaches one or both of the flexible sheath and the catheter to the control handle in a bayonet-style connection.

In some implementations, the catheter has at least one outwardly-projecting pin or dimple near a distal end thereof which extends outward into a helical or serpentine channel of the flexible sheath. In some implementations, relative rotation of the catheter and the flexible sheath causes relative axial displacement therebetween.

In some implementations, there are two dimples extending on opposite sides of the catheter that project into complementary helical or serpentine channels on the flexible sheath.

In some implementations, the system comprises a primary stabilization system configured to hold stable the control handle. In some implementations, the primary stabilization system includes a clamp assembly with movable jaws that hold a portion of the control handle. In some implementations, the system also includes a secondary stabilization system configured to hold stable a distal end of the flexible sheath.

In some implementations, the system comprises a crimping assembly arranged or arrangeable around or relative to an expandable device. In some implementations, the crimping assembly is configured to simultaneously radially crimp and axially displace the device in a proximal direction.

In some implementations, actuation of the crimping assembly facilitates crimping and displacement of the expandable device into the catheter from an initial expanded state.

In some implementations, an intermediate tube has a lumen through which an inner tube passes. In some implementations, the expandable device is positioned or positionable around the inner tube. In some implementations, the intermediate tube is sized to slide through the catheter (e.g., a distal portion of the catheter, etc.).

In some implementations, the intermediate tube terminates in a retention member that grasps a proximal end of the expandable device. In some implementations, proximal movement of the intermediate tube relative to the catheter pulls the expandable device within the catheter.

In some implementations, the intermediate tube has a fiducial marking along its length. In some implementations, the catheter has a positioning window. In some implementations, the positioning window is placed close to the control handle. In some implementations, a particular position of the fiducial marking visible through the positioning window indicates proper positioning of the expandable device within the catheter.

In some implementations, the system comprises a tapered nose cone coupled to and projecting distally relative to a distal end of the catheter. In some implementations, the nose cone is adapted to facilitate passage of the delivery catheter through vasculature of the subject. In some implementations, a first array of light emitters are distributed around the nose cone. In some implementations, the light emitters are connected to fiber-optic lines extending between the control handle and the nose cone to supply energy (e.g., light energy, etc.) to the emitters. In some implementations, a second array of optical receptors are distributed around the nose cone and spaced from the first array.

In some implementations, the system comprises an access hub with a plurality of fluid seals along an internal passage. In some implementations, the access hub is fixed or fixable to the flexible sheath. In some implementations, the flexible sheath sized to pass through the internal passage in a sealed manner (which is one way the access hub can be fixed to the flexible sheath). In some implementations, the flexible sheath can pass through the internal passage in a sealed manner, through an incision in a subject, and into a blood vessel of the subject.

In some implementations, a docking station secured adjacent or proximate the incision. In some implementations, the docking station is secured adjacent or proximate the incision using a strap sized to extend around a leg of the subject. In some implementations, the docking station has a tubular dock sized and shaped to receive and stabilize the access hub outside the incision.

In some implementations, an intermediate jacket is sized and shaped to conform around the access hub and having an exterior shape sized and shaped to form an interference fit within the dock.

In some implementations, transcatheter delivery systems with features for facilitating improved advancement within the body are disclosed. In some implementations, one or more transcatheter delivery systems herein can include an intelligent nose cone. In some implementations, the intelligent nose cone may include a visualization or sensory system for detecting when the nose cone is in contact with surrounding tissue (e.g., which can in some circumstances indicate rerouting is required).

In some implementations, transcatheter delivery systems can comprise concentric tubes in a delivery system sheath assembly. In some implementations, the concentric tubes can have one or more cooperating structures such that rotation of a first tube of the concentric tubes causes axial displacement of a second tube of the concentric tubes. In some implementations, this facilitates fine movement of the distal end and/or a device (e.g., a heart valve, an implant, a treatment device, a repair device, etc.) therein.

In some implementations, improved bendable sections are also disclosed which facilitate greater degrees of flexibility. In some implementations, the bendable sections can be configured to enable a user to convert the delivery system sheath between rigid and flexible configurations.

In some implementations, a delivery system which is detachable from the control handle enables reuse of the control handle which can then be made of higher quality materials that can be sterilized.

In some implementations, features for stabilizing and manipulating the delivery system during advancement of the heart valve into the body, as well as when loading the heart valve are also disclosed.

In some implementations, an example delivery system (e.g., a device delivery system, a treatment device delivery system, an implant delivery system, a prosthetic heart valve delivery system, etc.) comprises a proximal control handle and a flexible access sheath attached to the control handle. In some implementations, the delivery system comprises an elongated sheath portion with a lumen.

In some implementations, the sheath portion includes a distal segment extending from a first distal ring to a first proximal ring with a length of flexible tubing therebetween.

In some implementations, a proximal segment is in series with a distal segment extends from a second distal ring to a second proximal ring with a length of flexible tubing therebetween.

In some implementations, a delivery catheter extends distally from or distally relative to the control handle and has an outer diameter sized to fit through the lumen of the sheath portion. In some implementations, the delivery catheter also having a lumen (e.g., delivery catheter lumen) extending therethrough.

In some implementations, a device (e.g., a prosthetic device, an implant, a heart valve, a treatment device, etc.) is expandable and is adapted to be collapsed (e.g., radially collapsed, laterally collapsed, etc.) and positioned within the delivery catheter lumen along a distal end portion of the delivery catheter lumen.

In some implementations, a plurality of pull wires extend distally from actuators within the control handle. In some implementations, a set of pull wires (e.g., a first set of pull wires) extends through axial channels in the first proximal ring and second distal ring to be anchored to the first distal ring. In some implementations, a set of pull wires (e.g., a second set of pull wires) extend to and/or are anchored to the second distal ring. In some implementations, the first and second sets of pull wires are configured such that when placed in tension by the actuators in the control handle, they bend either or both of the distal segment and proximal segment in one or more of a plurality of directions.

In some implementations, the lengths of flexible tubing may comprise Nitinol tube having laser-formed circumferential cuts therein.

In some implementations, the first proximal ring may be conjoined (or otherwise coupled) with the second distal ring to form a single intermediate ring.

In some implementations, the first set of pull wires may be anchored to the first distal ring by T-shaped grommets that fit within similarly shaped and sized slots in the first distal ring.

In some implementations, there may be three wires in the first set of pull wires angularly spaced 120° apart. In some implementations, there may be three wires in the second set of pull wires angularly spaced 120° apart and offset from the wires in the first set of pull wires by 60°.

In some implementations, each of the pull wires is independently actuated from the control handle.

In some implementations, a delivery system (e.g., a device delivery system, an implant delivery system, a treatment device delivery system, a prosthetic heart valve delivery system, etc.) comprises a proximal control handle and a flexible access sheath attached to the control handle. In some implementations, the flexible access sheath comprises an elongated sheath portion with a lumen.

In some implementations, the sheath portion includes a bendable segment terminating in a distal ring. In some implementations, the bendable segment has a proximal segment connected in series with a distal segment.

In some implementations, the proximal segment comprises a series of solid ribs that are flexibly connected together for bending and having larger gaps therebetween on a lower circumferential side than on an upper side. In some implementations, the distal segment comprises a series of solid ribs that are flexibly connected together for bending and having larger gaps therebetween on an upper circumferential side than on a lower side.

In some implementations, a delivery catheter extends distally from or distally relative to the control handle and has an outer diameter sized to fit through the lumen of the sheath portion, the delivery catheter also having a lumen extending therethrough. In some implementations, an expandable device (e.g., a prosthetic heart valve, an implant, a treatment device, a repair device, etc.) is adapted to be collapsed (e.g., radially collapsed, laterally collapsed, etc.) and positioned within the delivery catheter lumen along a distal end portion thereof.

In some implementations, a plurality of pull wires extends distally from actuators within the control handle. In some implementations, a first pull wire extends through the bendable segment and being anchored to an upper circumferential side of the distal ring such that an actuator in the control handle can place the first pull wire in tension and bend the proximal segment downward. In some implementations, a second pull wire extends through the bendable segment and being anchored to a lower circumferential side of the distal ring such that, such that an actuator in the control handle can place the second pull wire in tension and bend the distal segment upward.

In some implementations, the distal and proximal segments may be adapted to bend in opposite directions in only one plane. In some implementations, the distal segment may be shorter than the proximal segment. In some implementations, the proximal and distal segments may be formed from a solid Nitinol tube with cuts. In some implementations, the proximal and distal segments may comprise helically arranged coils with relatively tighter and looser coil spacing.

In some implementations, a delivery system (e.g., a device delivery system, a treatment device delivery system, an implant delivery system, a repair device delivery system, a prosthetic heart valve delivery system, etc.) comprises a flexible access sheath having an elongated sheath portion with a lumen.

In some implementations, the sheath portion includes a bendable segment having an inner tube comprising a series of solid ribs that are flexibly connected together for bending and having larger gaps therebetween on one circumferential side than on an opposite side. In some implementations, the bendable segment also has an outer tube comprising a series of solid ribs that are flexibly connected together for bending and having larger gaps therebetween on one circumferential side than on an opposite side.

In some implementations, a proximal control handle attached to the control handle has an actuator for rotating the inner tube relative to the outer tube, or vice versa.

In some implementations, a delivery catheter extends distally from or distally relative to the control handle and has an outer diameter sized to fit through the lumen of the sheath portion. In some implementations, the delivery catheter has a lumen extending therethrough. In some implementations, an expandable device (e.g., a prosthetic heart valve, an implant, a treatment device, a repair device, etc.) is adapted to be collapsed and positioned within the delivery catheter lumen along a distal end portion thereof.

In some implementations, the actuator in the control handle can rotate the inner tube relative to the outer tube, or vice versa, to alternately align the larger gaps in the inner tube and outer tube and permit bending or misalign the larger gaps in the inner tube and outer tube and impede bending. In some implementations, the width and spacing of the larger gaps in the inner tube and outer tube may be equivalent.

In some implementations, a delivery system, such as a detachable prosthetic heart valve delivery system, comprises a proximal control handle having a plurality of annular members therein rotated by external actuators thereon.

In some implementations, a delivery system comprises a flexible access sheath extending distally from or distally relative to the control handle having a lumen. In some implementations, at least two pull wires attach at different axial locations within the access sheath for causing bending thereto.

In some implementations, a delivery catheter extends distally from or distally relative to a control handle and has an outer diameter sized to fit through the lumen of the sheath portion. In some implementations, the delivery catheter has a lumen extending therethrough.

In some implementations, an expandable device is adapted to be collapsed (e.g., radially collapsed, laterally collapsed, etc.) and positioned within the delivery catheter lumen along a distal end portion thereof. In some implementations, an expandable prosthetic heart valve is adapted to be collapsed (e.g., radially collapsed, laterally collapsed, etc.) and positioned within the delivery catheter lumen along a distal end portion thereof.

In some implementations, a tapered nose cone couples to and projects distally from or distally relative to a distal end of the delivery catheter. In some implementations, the nose cone is adapted to facilitate passage of the delivery catheter through the subject's vasculature. In some implementations, an inner tube extends through the lumen of the delivery catheter, through the prosthetic heart valve, and attaches to the nose cone.

In some implementations, the delivery system has an attachable/detachable control handle. In some implementations, the delivery system has a plurality of carriages, e.g., one for each of the delivery catheter, the at least two pull wires, and/or the nose cone.

In some implementations, the control handle is attachable to place (or such that it places) the annular members into engagement with the carriages and enable axial displacement of the delivery catheter, the at least two pull wires, and the nose cone.

In some implementations, in a delivery system (e.g., in a detachable prosthetic heart valve delivery system, a device delivery system, etc.), the access sheath may terminate at a proximal end in a hub having a pair of radially-outward lugs thereon.

In some implementations, a distal end of the control handle has a hollow cylindrical housing defining two notches sized and positioned to receive the lugs, such that insertion and rotation of the lugs through the notches of the housing attaches the control handle to the rest of the delivery system (or to one or more other components of the delivery system) in a bayonet-style connection.

In some implementations, in the delivery system (e.g., in a detachable prosthetic heart valve delivery system, in a device delivery system, etc.), the carriages each can have outer threading thereon. In some implementations, the annular members have internal threading to engage the outer threading on respective carriages to displace them axially.

In some implementations, each of the annular members can have split halves that can be retracted outward and then displaced inward to engage the outer threading on the carriages.

In some implementations, a system (e.g., an implant system, a treatment device system, a repair device system, a prosthetic heart valve system, etc.) comprises a proximal control handle having a plurality of external actuators thereon.

In some implementations, a flexible outer sheath or catheter extends distally from or distally relative to the control handle and has a lumen.

In some implementations, a delivery catheter has an outer diameter sized to fit through a lumen of an outer sheath that extends from the control handle. In some implementations, the delivery catheter is also having a lumen extending therethrough. In some implementations, the delivery catheter is linearly displaceable using one of the actuators on the control handle.

In some implementations, an expandable device (e.g., an expandable prosthetic heart valve, an expandable repair device, an expandable treatment device, an expandable implant, etc.) is adapted to be collapsed (e.g., radially collapsed, laterally collapsed, etc.) and positioned within the delivery catheter lumen along a distal end portion thereof.

In some implementations, a support mount is configured to sit on a raised table or platform and hold stable the control handle. In some implementations, the support mount includes a clamp assembly with movable jaws that surround a tubular portion of the control handle. In some implementations, the tubular portion of the control handle comprises an externally threaded or ribbed portion.

In some implementations, a rotational adjustment bolt is arranged to rotate about its axis while being held in a stationary position by the clamp assembly. In some implementations, the rotational adjustment bolt has threads that engage the threaded or ribbed portion of the tubular portion of the control handle. In some implementations, rotation of the rotational adjustment bolt rotates the control handle about its axis.

In some implementations, the clamp assembly can include a control knob for tightening and loosening the movable jaws in addition to the rotational adjustment bolt. In some implementations, the clamp assembly has a toggle-like latch that closes the movable jaws and provides positional stability without creating a tight interference fit. In some implementations, the rotational adjustment bolt can rotate the control handle when the latch is closed. In some implementations, the rotational adjustment bolt may have a wingnut-type outer end for ease of grasping.

In some implementations, a delivery system (e.g., a rotatable delivery system, a device delivery system, a treatment device delivery system, a repair device delivery system, a prosthetic heart valve delivery system, etc.) comprises a proximal control handle. In some implementations, the delivery system comprises a flexible access sheath or catheter extending distally from or distally relative to the control handle having an elongated sheath/catheter portion with a lumen.

In some implementations, a delivery catheter extending distally from or distally relative to the control handle has an outer diameter sized to fit through the lumen of the sheath portion. In some implementations, the delivery catheter has an actuator for rotating the delivery catheter with respect to the sheath portion. In some implementations, the delivery catheter is formed with or comprises a lumen extending therethrough.

In some implementations, an expandable device (e.g., an expandable treatment device, an expandable implant, an expandable prosthetic heart valve, an expandable repair device, etc.) is adapted to be collapsed (e.g., radially collapsed, laterally collapsed, etc.) and positioned within the delivery catheter lumen along a distal end portion thereof.

In some implementations, the delivery catheter has at least one outwardly-projecting pin or dimple near a distal end thereof which extends outward into a helical or serpentine channel formed in the sheath portion. In some implementations, relative rotation of the delivery catheter and sheath portion causes relative axial displacement therebetween. In some implementations, there may be two dimples extending on opposite sides of the catheter that project into complementary helical or serpentine channels on the sheath portion.

In some implementations, the delivery catheter may be driven by a rotatable flex cable from the control handle.

In some implementations, a delivery system (e.g., a device delivery system, treatment device delivery system, a repair device delivery system, a prosthetic heart valve delivery system, etc.) comprises a proximal control handle comprising a plurality of actuators (e.g., external actuators on the control handle, etc.). In some implementations, a flexible outer sheath extends distally from or distally relative to the control handle and has a lumen.

In some implementations, a delivery catheter has an outer diameter sized to fit through the lumen of the outer sheath. In some implementations, the delivery catheter also having a lumen extending therethrough. In some implementations, the delivery catheter is linearly displaceable using one of the actuators on the control handle.

In some implementations, an expandable device (e.g., an expandable treatment device, an expandable implant, an expandable prosthetic heart valve, an expandable repair device, etc.) is adapted to be collapsed (e.g., radially collapsed, laterally collapsed, etc.) and positioned within the delivery catheter lumen along a distal end portion thereof.

In some implementations, an inner tube extends distally from or distally relative to the control handle and through and beyond the delivery catheter lumen. In some implementations, a primary support mount is configured to sit on a raised table or platform and/or to hold stable the control handle. In some implementations, the primary support mount includes a clamp assembly with movable jaws that can surround a tubular portion of the control handle.

In some implementations, a secondary support mount is configured to sit on a raised table or platform and/or to hold stable a distal end of the outer sheath.

In some implementations, a crimping assembly is arranged or is arrangeable around the device (e.g., treatment device, heart valve, implant, etc.). In some implementations, the crimping assembly is configured to simultaneously radially crimp and axially displace the device in a proximal direction.

In some implementations, the crimping assembly and device in an expanded state can be initially positioned around the inner tube and distal to the delivery catheter. In some implementations, the crimping assembly is configured such that actuation of the crimping assembly facilitates crimping and displacement of the heart valve into the delivery catheter.

In some implementations, the system can optionally include an intermediate tube having a lumen through which the inner tube passes. In some implementations, the intermediate tube is sized to slide through the delivery catheter. In some implementations, the intermediate tube can terminate in a retention member that grasps a proximal end of the device (e.g., of a heart valve, implant, etc.). In some implementations, the system can be configured such that proximal movement of the intermediate tube relative to the delivery catheter pulls the heart valve within the delivery catheter.

In some implementations, the intermediate tube can also have a fiducial marking along its length. In some implementations, the delivery catheter has a positioning window placed close to the control handle. In some implementations, a particular position of the fiducial marking is visible through the positioning window to indicate proper positioning of the heart valve within the delivery catheter.

In some implementations, a delivery system (e.g., a device delivery system, treatment device delivery system, a repair device delivery system, a prosthetic heart valve delivery system, etc.) comprises a proximal control handle. In some implementations, a flexible access sheath extends distally from or distally relative to the control handle and has a lumen.

In some implementations, a delivery catheter extends distally from or distally relative to the control handle. In some implementations, the delivery catheter has an outer diameter sized to fit through the lumen of the access sheath. In some implementations, the delivery catheter is formed with or comprises a lumen extending therethrough.

In some implementations, an expandable device (e.g., an expandable treatment device, an expandable implant, an expandable prosthetic heart valve, an expandable repair device, etc.) is adapted to be collapsed (e.g., radially collapsed, laterally collapsed, etc.) and positioned within the delivery catheter lumen along a distal end portion thereof.

In some implementations, a tapered nose cone is coupled (e.g., directly or indirectly) to and projects distally from or distally relative to a distal end of the delivery catheter. In some implementations, the nose cone is adapted or configured to facilitate passage of the delivery catheter through vasculature of a subject (e.g., a living subject, a simulation, etc.).

In some implementations, the nose cone comprises and/or is coupled to an array of light emitters (e.g., a first array, a first array of light emitters, etc.). In some implementations, the light emitters are distributed around the nose cone. In some implementations, the light emitters are coupled or connected to fiber-optic lines extending through the delivery system to supply light energy to the emitters (e.g., extending from the proximal control handle, from a proximal location, from outside the subject, etc.).

In some implementations, the nose cone comprises and/or is coupled to an array of optical receptors (e.g., a second array, etc.). In some implementations, the array of optical receptors comprises circumferentially-spaced optical receptors. In some implementations, optical receptors are distributed around the nose cone. In some implementations, the optical receptors are axially spaced from the array of light emitters (or from the first array, etc.). In some implementations, the array of light emitters can be distributed in a circumferential pattern.

In some implementations, the first array can be located closer to a proximal end of the nose cone than a distal tip. In some implementations, the optical emitters can be LEDs.

In some implementations, a delivery system (e.g., a device delivery system, treatment device delivery system, a repair device delivery system, a prosthetic heart valve delivery system, etc.) comprises a docking station. In some implementations, the delivery system comprises a proximal control handle. In some implementations, the delivery system comprises a flexible outer sheath extending distally from or distally relative to the control handle having a lumen.

In some implementations, a delivery catheter extends distally from or distally relative to the control handle. In some implementations, the delivery catheter has an outer diameter sized to fit through the lumen of the access sheath. In some implementations, the delivery catheter has a lumen extending therethrough.

In some implementations, an expandable device (e.g., an expandable prosthetic heart valve, an expandable treatment device, an expandable repair device, an expandable implant, an expandable valve, etc.) is adapted to be collapsed (e.g., radially collapsed, laterally collapsed, etc.) and positioned within the delivery catheter lumen along a distal end portion thereof.

In some implementations, a system (e.g., any of the systems, delivery systems, etc. described above or elsewhere herein) has an access hub with a plurality of fluid seals along an internal passage. In some implementations, the access hub is fixed to a distally-extending access sheath sized to pass through an incision in a subject (e.g., a living subject, a simulation, etc.) and into a blood vessel of the subject.

In some implementations, the outer sheath is sized to pass through the internal passage in a sealed manner and into the blood vessel.

In some implementations, a docking station is secured or configured to be secured adjacent an incision using a strap sized to extend around part of a subject's body (e.g., around a subject's limb, around a subject's leg, etc.). In some implementations, the docking station has a tubular dock sized and shaped (e.g., configured) to receive and stabilize the access hub immediately outside the incision.

In some implementations, the system can include an intermediate jacket sized and shaped to conform around the access hub. In some implementations, the intermediate jacket has an exterior shape sized and shaped (e.g., configured) to form an interference fit within the dock. In some implementations, the jacket may a ferromagnetic material, and the dock is magnetized.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 is a schematic representation of a transcatheter mitral procedure showing advancement of a catheter or sheath through a blood vessel in the leg up to the vicinity of the mitral valve within the heart, and FIG. 1A is an enlargement of the heart showing the distal end of the catheter or sheath in a potential approach to a tricuspid valve;

FIGS. 2A-2D are cross-sections of a heart showing a sequence of steps in a mitral valve replacement procedure utilizing a prosthetic valve delivery system;

FIG. 3 is a longitudinal sectional view through the distal section of a prosthetic mitral valve delivery system having a leading nose cone, and FIG. 3A is a radial sectional view taken along line 3A-3A of FIG. 3 through a crimped prosthetic mitral valve held within the prosthetic mitral valve delivery system;

FIG. 4 is an elevational view of an example nose cone having optical emitters and receptors for use with a delivery system, such as the valve delivery system of FIG. 3;

FIG. 5A illustrates use of the modified nose cone of FIG. 4 showing optical feedback indicating proper advancement of the delivery system, and FIG. 5B illustrates a situation where the nose cone is in improper contact with a surrounding tissue wall which forecloses optical feedback indicating improper advancement;

FIG. 6A is a schematic view of a transcatheter delivery system held by user, and FIG. 6B is an enlarged view of a sheath portion thereof showing an expanded transcatheter device;

FIG. 7 is an enlargement of a distal bendable end of the sheath portion of the delivery system in the process of transcatheter device expansion from a distal end thereof;

FIG. 8 is an example distal bendable end of the access sheath as in FIG. 7 illustrating one solution for making fine axial adjustments to an inner catheter thereof;

FIG. 9A is an isolated section of two cooperating concentric tubes of the access sheath of FIG. 8, and FIG. 9B shows a section of just the inner catheter tube;

FIG. 10 is a perspective view of a bendable section of a sheath portion of a valve delivery system;

FIGS. 11A and 11B are perspective views of example inner pull wires used within the bendable section of FIG. 10;

FIG. 12 is an elevational view of an example bendable section of a sheath portion of a valve delivery system;

FIGS. 13A-13C show different ways the bendable section of FIG. 12 can flex;

FIG. 14A is a partially cutaway elevational view of a section of an example bendable section of the sheath portion of a valve delivery system having concentric tubes that can be aligned for bending as shown, and FIGS. 14B and 14C show the same concentric tubes having been rotationally misaligned to inhibit bending;

FIG. 15 is an elevational view of an example delivery system, and FIG. 15A is an enlargement of a proximal control handle thereof;

FIG. 16 is a perspective view of a distal end of the example delivery system of FIG. 15 following distal displacement of a distal nose cone, and FIG. 16A is a sectional view thereof;

FIG. 17 is a perspective view of the distal end of the example delivery system following proximal displacement of an outer sheath to expose a prosthetic device, and FIG. 17A is a sectional view thereof;

FIG. 18 is a perspective view of the distal end of the example delivery system following proximal displacement of an intermediate sheath to expose and allow expansion of a device, and FIG. 18A is a sectional view thereof;

FIG. 19 is a perspective view of the distal end of the example delivery system illustrating bending capacity of an intermediate sheath, and FIG. 19A is a sectional view of a portion thereof;

FIG. 20A is an elevational view of an example delivery system control handle of the present application indicating a number of control dials being rotated, and FIG. 20B is a partial sectional view illustrating engagement between an example sheath assembly and the control handle;

FIG. 21A is an exploded perspective view of a distal end of the control handle of FIG. 20A adjacent a proximal end of the sheath assembly, and FIG. 21B shows the two components being coupled together using a bayonet-type lock;

FIG. 22 is an exploded view of the example delivery system of FIG. 20A;

FIGS. 23A and 23B are isolated views of the example sheath assembly showing two possible modes of operation thereof;

FIG. 24 is a perspective view of a proximal end of an example delivery system showing a control handle and access hub being held in an example support mount;

FIG. 25A is a perspective view of a clamp region of the example support mount showing a rotational adjustment bolt, and FIG. 25B is a perspective view with only a portion of the delivery system shown with which the rotational adjustment bolt interacts;

FIG. 26 is a perspective view of an example clamp of an example support mount indicating another configuration for rotational adjustment of the delivery system;

FIG. 27 is a side elevational view of an example assembly of a delivery system with a proximal end held on an example primary support mount, as described above, and a distal end held by an example secondary support mount to enable one-handed valve loading, and FIG. 27A is an enlargement of a proximal portion of an outer sheath illustrating a positioning window therein;

FIG. 28 is a side elevational view of a distal end of the assembly of FIG. 28, and FIG. 29 is an enlarged view of a distal end of the delivery system showing a crimper assembly, and FIG. 29A is an enlargement of a retention member;

FIGS. 30A and 30B are sectional views through the distal end of the delivery system of FIG. 28 showing crimping and loading of a device into the access sheath;

FIGS. 31A and 31B are side elevational views of the positioning window on the proximal portion of the outer sheath during the steps illustrated in FIGS. 30A and 30B, respectively;

FIG. 32 is a schematic representation of a transcatheter procedure, as in FIG. 1, and FIG. 32A is an enlargement of an example method for securing an access hub at the incision using sutures; and

FIG. 33 is a perspective view of an upper leg of a subject illustrating an example hub docking station, and FIG. 33A is an exploded enlargement thereof.

### DETAILED DESCRIPTION

The right ventricle and left ventricle are separated from the right atrium and left atrium, respectively, by the tricuspid valve and mitral valve; i.e., the atrioventricular valves. The septal wall extends between the right atrium and left atrium. The present specification and drawings provide aspects and features of the disclosure in the context of several implementations of replacement heart valves, delivery systems and methods that are configured for use in the vasculature of a subject (e.g., a living subject, a simulation, etc.), such as for treatment (such as replacement, repair, etc.) of natural heart valves in a subject. Valve treatment of the mitral or tricuspid valves is a primary focus of the examples in the present application, but characteristics of the delivery systems described herein may equally be used for other valve locations, and thus the claims should not be constrained to only mitral or tricuspid valve replacement unless expressly limited.

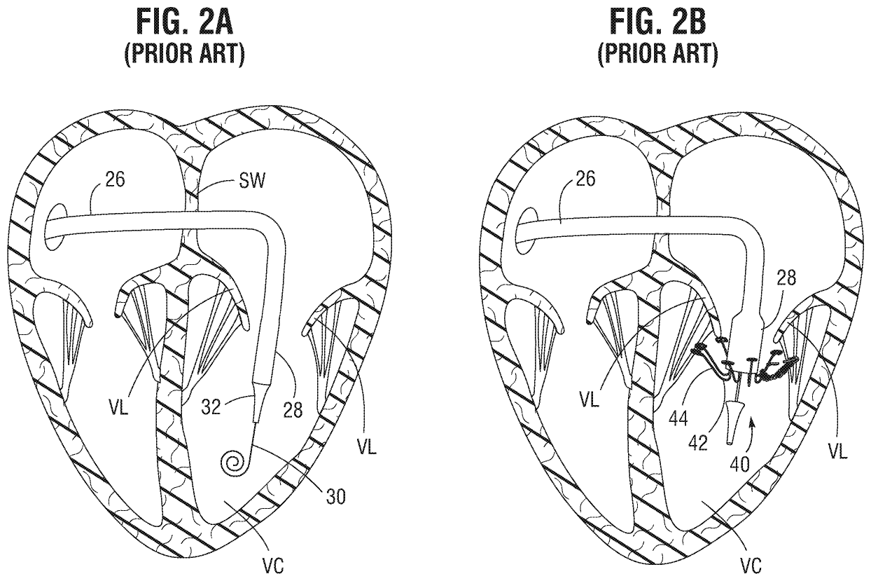

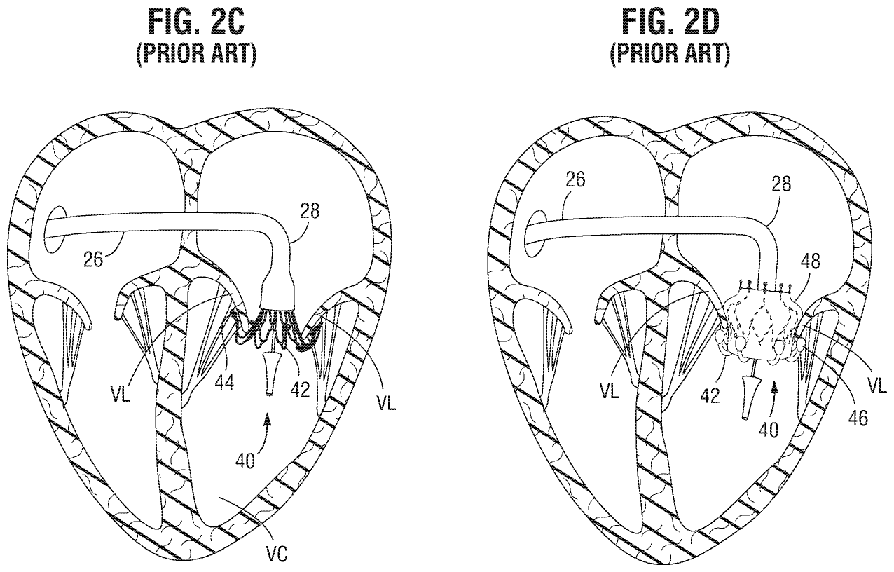

In some implementations, delivery systems described herein can be used for transfemoral percutaneous delivery of a treatment device, such as a replacement mitral valve or a repair device, to a mitral valve to treat patients with moderate to severe mitral regurgitation (or to a tricuspid valve to treat moderate to severe tricuspid regurgitation). In some cases, for safety and/or other reasons, the disclosed devices may be delivered from the atrial side of the atrioventricular valve or atrioventricular valve annulus. For example, a transatrial approach can be made through an atrial wall, which can be accessed, for example, by an incision through the chest. Atrial delivery can also be made intravascularly, such as from a pulmonary vein. The prosthetic valve can be delivered to the right atrium via the inferior or superior vena cava. In some cases, left atrial delivery can be made via a transeptal approach (e.g., FIGS. 2A-2D). In a transeptal approach, an incision can be made in the atrial portion of the septal wall SW to allow access to the left atrium from the right atrium (the septum can be approached in one or more of a variety of transcatheter approaches, e.g., transjugularly, transfemorally, etc.). In short, the prosthetic valve can be delivered via transventricular, transatrial, transjugular, and/or transfemoral approaches with small or minimal modifications to the delivery process. Any of the approaches that travel through any vasculature can be call a transvascular approach as well.

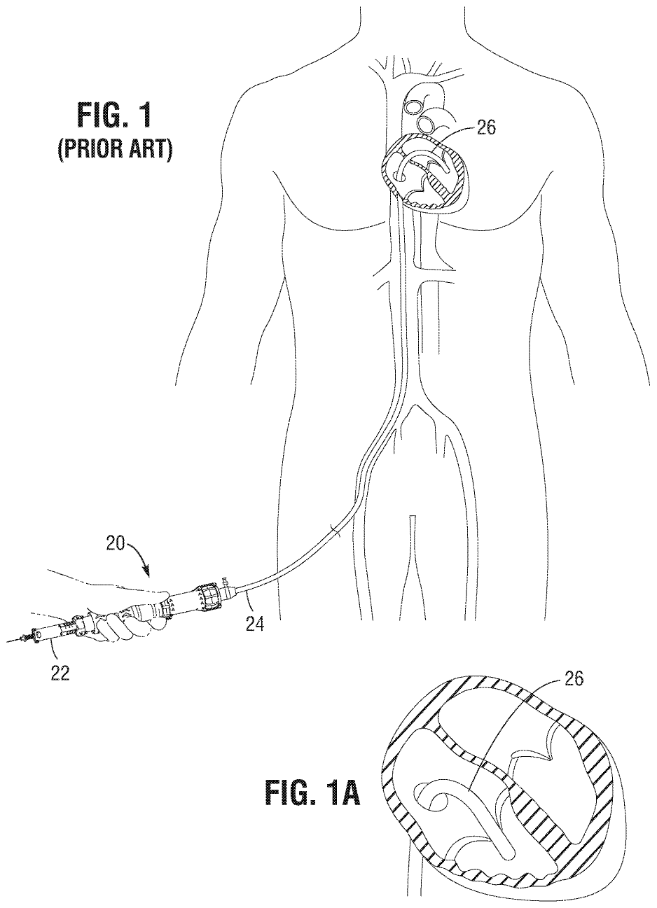

FIG. 1 illustrates a transcatheter mitral valve replacement procedure showing advancement of a delivery system 20 through a blood vessel in the leg such as the femoral vein up to the vicinity of the mitral valve within the heart. Example transfemoral approaches that can be used with the various implementations and inventive concepts herein can be found in U.S. Pat. Nos. 10,004,599 and 10,813,757, the entireties of which are hereby incorporated by reference for all purposes, but again, a variety of approaches are possible.

The illustrated prosthetic mitral valve delivery system 20 has a proximal handle 22 from which an elongated access sheath 24 extends distally. The access sheath 24 is shown extending into a lower portion of the venous system, such as into an ipsilateral femoral vein, and a catheter sheath 26 advances from within the access sheath 24 up through the patient's venous system into the right atrium to access the tricuspid valve, or with a further transseptal puncture using known techniques into the left atrium to access the mitral valve. FIG. 1 shows the latter procedure where a distal tip 28 (see FIG. 2A) of the catheter sheath 26 has crossed the septal wall SW. FIG. 1A, in contrast, shows a potential approach to access the tricuspid valve with the distal tip of a delivery system in the right atrium directed to the tricuspid valve. Both procedures can be enhanced by the various delivery system attributes described herein, individually or in conjunction with one another. All combinations of structural features as described herein are contemplated, assuming they are not mutually exclusive or redundant in deployment.

In some implementations herein, the access sheath 24 can be integrally associated with the proximal handle 22 or can be a separate instrument. In some implementations, an integral sheath 24 would be fixedly attached to the proximal handle 22, with the catheter sheath 26 extending through and movable with respect to both the handle 22 and the sheath. With a separate sheath 24, in some implementations, the sheath can have a proximal hub with elastomeric valves and/or seals. In some implementations, the catheter sheath 26 can be fixedly attached to the proximal handle 22 and passed through the valves or seals to prevent blood leakage. Both types of access sheath 24 are contemplated herein, and the claims should not be considered limited to one or the other unless specifically recited.

The proximal handles (e.g., handle 22, etc.) herein as well as attendant support systems such as guidewires and inflation connections can be configured in a variety of ways, and can comprise one, some, or all of the features seen in U.S. Pat. Nos. 10,004,599 and 10,813,757, incorporated by reference herein, in addition to any features described with respect to various implementations herein.

To better understand certain aspects of the improvements disclosed herein, an example mitral valve replacement procedure utilizing an example valve delivery system 20 will be described with reference to FIGS. 2A-2D. The figures are labeled “PRIOR ART” relative to the basic steps illustrated. The same or similar steps or improved steps can be practiced with one or more of the inventive and new features/steps described herein with respect to the various implementations herein.

While some of the basic steps illustrated may be “PRIOR ART”, some of the discussion and descriptions below regarding FIGS. 1-3 may not be in the prior art, i.e., the descriptions and discussions below may include descriptions of steps and/or features that are improvements over the prior art. In other words, just because a step or feature is described below relative to one or more of FIGS. 1-3, does not necessarily mean it is in the prior art.

To deliver the prosthetic valve to the native mitral valve annulus, the prosthetic valve can be positioned within a catheter sheath 26 of the delivery system 20.

With reference to both FIGS. 1 and 2A, the access sheath 24 of the delivery system 20 can be placed in the ipsilateral femoral vein and the catheter sheath 26 advanced through the sheath toward the right atrium. A transseptal puncture through the septal wall SW using known techniques can then be performed to obtain access to the left atrium. The catheter sheath 26 can then be advanced into the left atrium and then to the left ventricle. In some implementations, a guidewire 30 can be used to position the catheter sheath 26 in the proper position, and one or more guidewires can be used. In some implementations, a generally conical or otherwise tapered nose cone 32 can be secured at the distal end 28 of the catheter sheath 26 to help transit through the vasculature and other obstacles, such as the septal wall SW, as well as through the valve leaflets VL into the associated ventricular cavity VC.

It can be advantageous for a user to be able to steer the delivery system 20 through the complex areas of the heart in order to position a replacement mitral valve in line with the native mitral valve. For example, ability to steer or bend one or more portions of the sheaths/catheters may allow a user to manipulate the distal end 28 of the catheter sheath 26 to the appropriate area. The delivery system 20 can be advanced through the transseptal puncture and into the left atrium and can then further manipulate the delivery system 20 to create an even greater bend in the catheter sheath 26. Further, a user can torque the entire delivery system 20 to further manipulate and control the position of the distal end 28. The catheter sheath 26 can be further advanced such that the catheter sheath 26 (carrying the prosthetic valve) extends between the native leaflets of the mitral valve and into the left ventricle VC.

FIGS. 2B-2C show an example prosthetic valve delivery, including a prosthetic valve 40 expanding as the catheter sheath 26 is retracted. FIG. 2B indicates partial retraction of the catheter sheath 26 to expulse a plurality of ventricular anchors 42 in a circumferential array from a distal end of the prosthetic valve.

In some implementations, the heart valve 40 can be displaced relative to the catheter sheath 26 such as by advancing a pusher device distally against the prosthetic valve and/or retracting the catheter sheath relative to the valve. In some implementations, terminal end portions 44 of the anchors 42 are biased to rotate to extend proximally (in the direction of a main body of the valve) when deployed. In some implementations, prior to rotation of the anchors 42, the constraining or restraining force applied by the catheter sheath 26 on the anchors 42 forces the terminal end portions 44 to extend downward (away from the main body in a generally distal direction) during delivery.

In some implementations, once the prosthetic valve 40 is delivered to the native annulus region, the catheter sheath 26 can be retracted farther relative to the prosthetic valve 40, thereby allowing the prosthetic valve 40 to expand radially outward. The release of the prosthetic valve 40 can be conducted in stages. In some implementations, the ventricular anchors 42 can be released from the catheter sheath 26 (FIG. 2B) prior to the release of a main body portion of the valve 40, as seen in FIG. 2C. In some implementations, when the ventricular anchors 42 are released, they spread out away from the main body, with distal end portions 44 directed radially outward and upward. Subsequently, with release of the main body, the anchors 42 rotate toward the main body, such that the distal end portions 44 pivot toward the vertical (longitudinal) axis and wrap around behind the native leaflets VL on the ventricular side.

Optionally, the delivery system can be manipulated/adjusted to reposition the partially retracted valve 40 as desired, and retracts the catheter sheath 26 further to cause the ventricular anchors 42 to engage the native valve annulus (FIG. 2C). Rounded head portions 46 of the ventricular anchors 42 can contact a ventricular side of the native valve annulus and/or adjacent tissue (such as trigone areas). In some implementations, the anchors 42 can be configured to point more directly upward upon full deployment, as compared to when they are partially deployed from the catheter sheath 26. At this point, the user can assess engagement of the ventricular anchors 42 with the native valve annulus (such as through imaging means), prior to retracting the catheter sheath 26 farther to deploy an atrial portion 48, as in FIG. 2D.

In some implementations, one or more ventricular anchors 42 can engage the chordae tendineae, one or more ventricular anchors can engage the trigone areas, and/or one or more ventricular anchors can engage the native leaflets at A2 and/or P2 positions (i.e., between the commissure of the native leaflets).

In some implementations, devices herein can include ventricular anchors that engage the native leaflets and the trigone areas can capture or “sandwich” the native tissue between the outer surface of the main body of the prosthetic valve and the ventricular anchors (or portions thereof) such that the tissue is compressed and engaged by the main body of the device on one side and by the ventricular anchors on the other side. In some implementations, due to capturing of the native tissue (such as the native leaflets) between the ventricular anchors and the main body, the native tissue may form a seal around the main body (e.g., through 360 degrees) within the left ventricle that impedes blood from traveling along the outside of the main body.

In some implementations, a relatively thin profile and that the ventricular anchors are not interconnected to each other may allow the distal ends of the ventricular anchors adjacent the chordae tendineae to pass between individual chords extending from the native leaflets, allowing those anchors to flex/pivot upwardly and assume their fully deployed positions.

In some implementations, as shown in FIG. 2D, the catheter sheath 26 can be retracted farther to release the atrial portion 48 of the prosthetic valve 40.

In some implementations, devices herein can be configured such that an atrial portion (such as atrial portion 48) forms a seal against the native annulus within the atrium. In some implementations, the device can be configured such that a seal created in the left atrium by the atrial portion 48 and the seal created by the ventricular anchors 42 in the left ventricle together prevent, reduce, or minimize the flow of blood between the native annulus and the outside of the main body during diastole and systole.

In some implementations, the main body and the atrial portion 48 are released simultaneously, while in some implementations, the main body is released prior to the atrial portion 48. In some implementations, upon full deployment of the ventricular anchors 42 and the main body, the distal end portions 44 can be positioned against the native valve annulus and/or adjacent tissue (e.g., trigone areas). In some implementations, all stages of deployment of the prosthetic valve 40 can thus be controlled by the catheter sheath 26 without additional activation or manipulation necessary.

In some implementations, the sheaths and/or catheters described with respect to the various implementations herein can be sized/configured to be a 30, 29 F, 28 F, 27 F, 26 F, etc. In some implementations, the various devices (e.g., treatment devices, repair devices, replacement devices, implants, prosthetic heart valves, etc.) herein can be configured to fit into sheaths and/or catheters of one or more sizes, such as a 30 F, 29 F, 28 F, 27 F, 26 F, etc. sheath/catheter.

Some leading nose cones, e.g., as seen at 32 in FIG. 2A, can by nature be somewhat sharp and elongated, which may create potential for improperly contacting anatomical structures and becoming stuck.

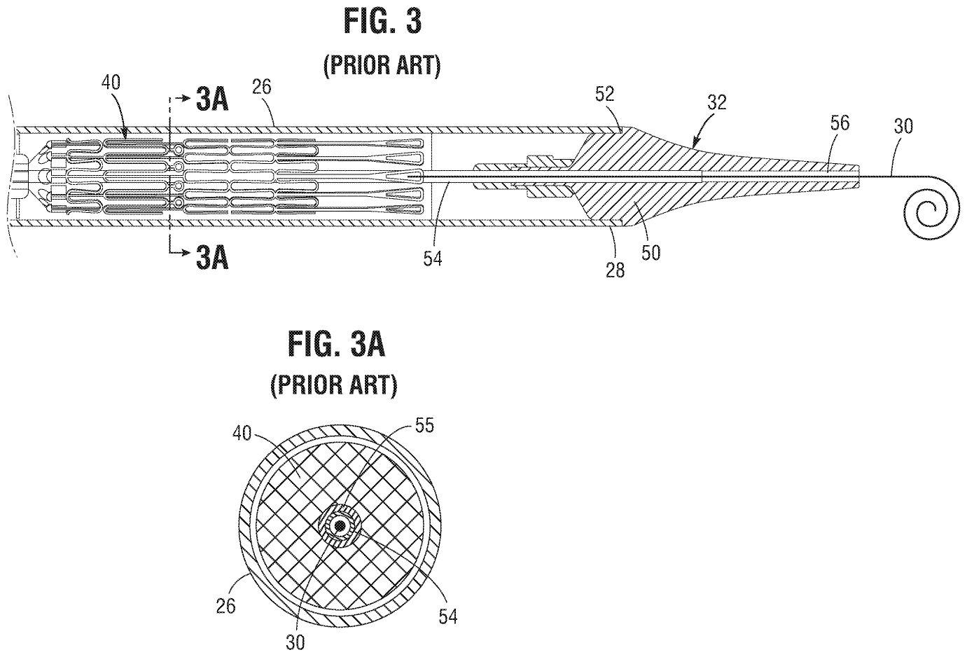

FIG. 3 schematically illustrates a longitudinal sectional view through an example catheter sheath 26. The nose cone 32 has an elongated, tapered front-end which widens to a proximal base portion 50. The base portion 50 can optionally include an exterior step 52 that mates with the distal end 28 of the sheath 26. In some implementations, the nose cone 32 engages the distal end 28 of the sheath 26 with an interference fit, and is further secured on the distal end via attachment to an elongated guidewire tube 54. In some implementations, a guidewire tube 54 can extend proximally through the entire delivery system 20 to the proximal handle 22. The guidewire tube 54 can be hollow for passage of a guidewire and can be fixedly secured within a through bore 56 formed within the nose cone 32 for continued passage of a guidewire therethrough. In some implementations, a collapsed prosthetic valve 40 can be within the sheath 26 just proximal to the nose cone 32. For the purpose of understanding and comparison, the discussion of implementations of nose cones below may use some of the same element numbering to aid explanation and description.

FIG. 3A is a radial sectional view taken along line 3A-3A of FIG. 3 through a crimped balloon-expandable prosthetic heart valve 40 held within a valve delivery system. Within a central bore through the valve 40 passes the aforementioned elongated guidewire tube 54 that can receive a guidewire 30.

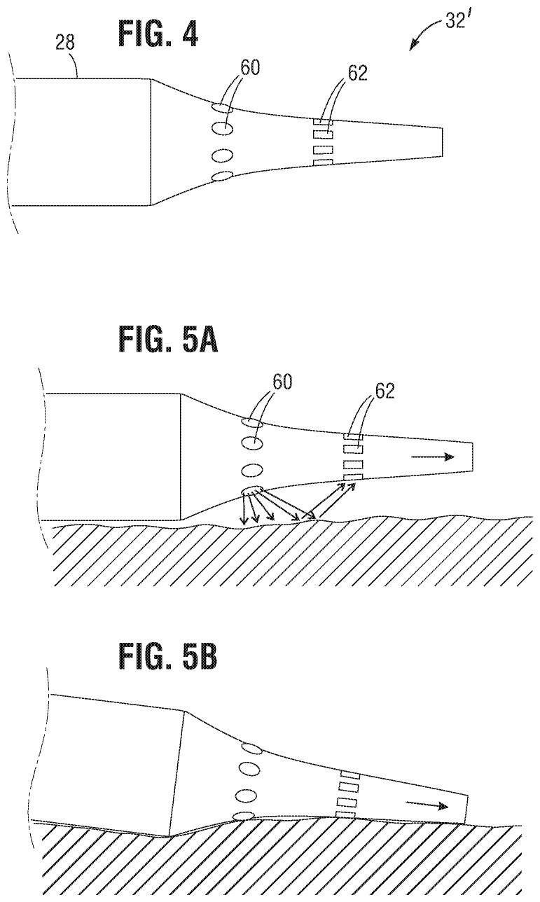

FIG. 4 is an elevational view of an example “smart” nose cone 32′ having optical emitters and receptors usable with various delivery systems (e.g., the valve delivery system of FIG. 3 and/or any of the other delivery systems described herein). In some implementations, the nose cone 32′ can include a series of emitters 60. In some implementations, the series of emitters 60 can be arrayed in a pattern (e.g., circumferential pattern, a linear pattern, a zig-zag pattern, a spiral pattern, etc.) on or around the nose cone 32′. In some implementations, the series of emitters 60 can be arranged in a circumferential pattern at a location that is closer to the proximal end of the nose cone than it is to a distal tip of the nose cone.

In some implementations, an array of optical receptors 62 is provided space from the array of emitters 60 (e.g., spaced distally from the array of emitters 60, spaced adjacent the array of emitters 60, spaced side-by-side with the array of emitters, etc.). The array of optical receptors can be arranged in a pattern similar to the array of emitters 60 or in a different pattern.

In some implementations, the optical emitters 60 may be LEDs. In some implementations, the optical receptors 62 may be photosensitive sensors which detect light. Although not shown, in some implementations, fiber-optic lines may extend from a proximal control handle through the delivery system to supply light energy to the emitters 60.

FIG. 5A illustrates use of the example nose cone 32′ of FIG. 4 showing optical feedback indicating proper advancement of the valve delivery system. In some implementations, the delivery system can be configured such that, if the delivery system is being advanced appropriately (e.g., straight, nearly straight, not angled relative to a portion of a vasculature wall, without being too close to a wall, etc.) through a blood vessel or other body cavity, the tapered portion of the nose cone 32′ will extend in space within the cavity, and the light rays from the emitters 60 will bounce off any surrounding wall and be sensed by the receptors 62. A feedback loop to control circuitry ensures the proper advancement.

However, FIG. 5B illustrates a situation where the nose cone 32′ is in contact with a surrounding tissue wall which obstructs optical feedback and indicates improper advancement (contact with a surrounding vessel wall or tissue). Namely, a portion of the spectrum of light generated by the emitter 60 will be interrupted before being sensed by the receptors 62 indicating an obstruction or abnormality in the path. This will trigger a warning signal by the control circuitry to the operator can then steer the catheter around the obstruction.

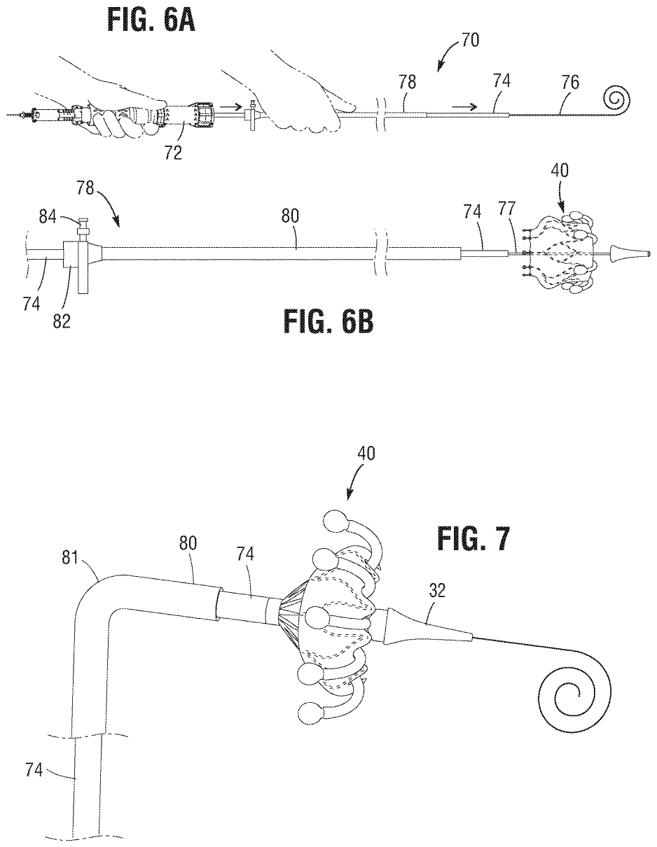

FIG. 6A is a schematic view of an example delivery system 70 (e.g., a device delivery system, a treatment device delivery system, a replacement device delivery system, a repair device delivery system, a prosthetic valve delivery system, etc.) held by a user, and FIG. 6B is an enlarged view of an access sheath 78 thereof showing a device 40 (illustrated as a transcatheter heart valve by way of example in the figure, but other devices are also possible, e.g., repair devices, implants, other configurations, etc.) expanded at a distal end thereof.

In some implementations, the system 70 has one, some, or all of a control handle 72 a catheter 74, a smaller inner tube 77 within the catheter 74, and/or a guidewire 76 within tube 77. In some implementations, each of the foregoing that are included can be arranged such that they are all concentric within the access sheath 78.

In one example operation/procedure, the user (e.g., a surgeon, cardiologist, technician, etc.) holds a portion of the access sheath 78 stable while distally advancing the control handle 72 and catheter 74. The inner tube 77 attaches to the nose cone 32, and may be displaced axially with respect to the catheter 74 by manipulation from the control handle 72. Additionally, the access sheath 78 may be bent in multiple places as it conforms to a tortuous anatomy, but has a certain amount of flexibility to navigate the blood vessels.

FIG. 7 illustrates an example access sheath which comprises an elongated flexible sheath portion 80 extending distally from or distally relative to a proximal hub 82 (see FIG. 6B). In some implementations, the hub 82 has one or more valves or seals along an internal passage to seal around the inner catheter 74 which concentrically slides through the sheath portion 80. In some implementations, a valve 84, which can be provided on the hub 82, can be configured to flush the system (e.g., a portion of the system, etc.) and/or to supply insufflation fluid (saline, air, or any fluid medium) to an inflatable filament or component. As will be seen below, the proximal hub 82 can optionally be integrated into a control handle, in which case the access sheath 78 can be fixedly attached to the control handle.

FIG. 7 is an enlargement of a distal bendable end of the sheath portion 80 of the delivery system 70 in the process of device expansion (e.g., expansion of an example transcatheter heart valve) from a distal end thereof. The inner catheter 74 may possess many capabilities to enable steering and fine positioning of the device 40 during the implant process. Thus, a right-angle bend 81 is seen in the catheter 74 and sheath portion.

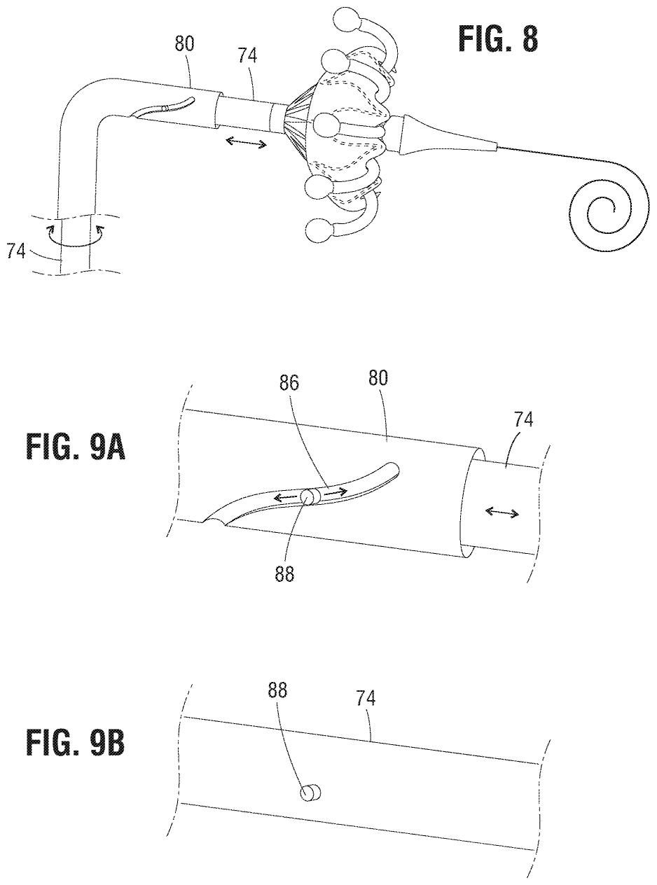

Some delivery systems may be limited in retraction if the device is placed too deep into the annulus, and thus FIG. 8 shows a modified access sheath 78 as in FIG. 7 with one solution for making fine axial adjustments to the inner catheter 74 carrying the device 40. FIG. 9A is an isolated section of the two cooperating concentric tubes of the access sheath 78, and FIG. 9B shows a section of just the inner catheter tube 74.

In some implementations, the sheath portion 80 has a helical or serpentine channel 86 formed therein on at least one side, preferably on opposite sides thereof. Likewise, in some implementations, the inner catheter 74 has an outwardly-projecting pin or dimple 88 extending outward therefrom and sized to slide within one of the channels 86. In some implementations, there are two dimples 88 extending on opposite sides of the catheter 74 that project into complementary helical channels 86 on the sheath portion 80.

In some implementations, by rotating the catheter tube 74, as indicated in FIG. 8, the dimple(s) 88 slide along the channel(s) 86 and cam the catheter tube 74 axially in either direction. The catheter 74 may be driven by a rotatable flex cable, and by rotating the flex cable, the catheter 74 can both translate forward and rotate the valve to provide additional features for a physician to place the device 40 correctly in the correct rotational position. Depending on the angle of the channel(s) 86, the axial distance relative to the angle of rotation can be calibrated for extremely fine adjustments.

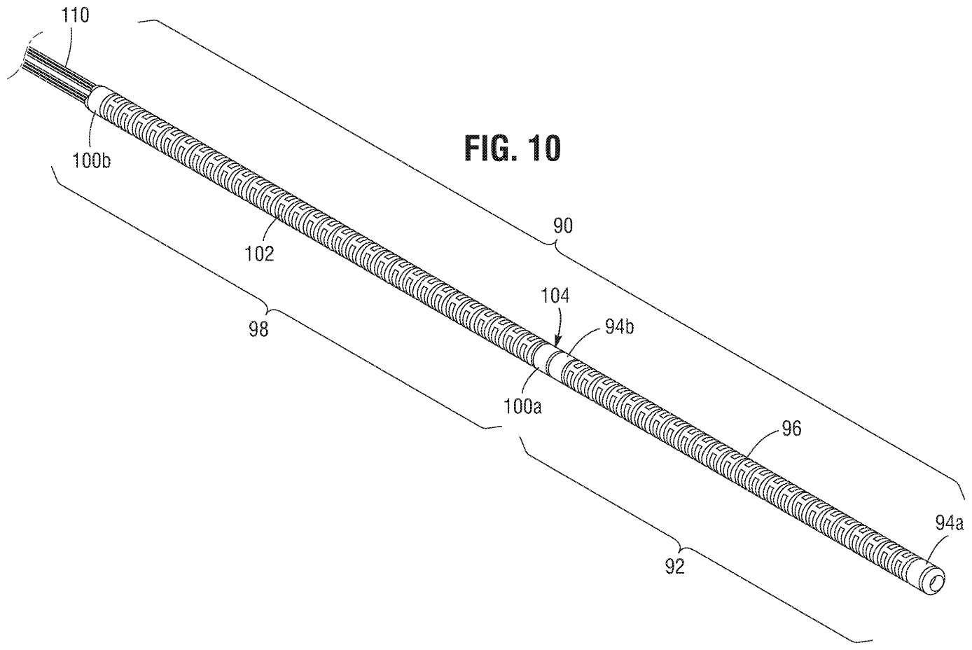

FIG. 10 is a perspective view of a bendable section 90 within a delivery system (e.g., a device delivery system, a treatment device delivery system, a repair device delivery system, an implant delivery system, a replacement device delivery system, a prosthetic valve delivery system, etc.). For context, the bendable section 90 is desirably tubular and can form or be formed as a portion of one of the tubes within the delivery system access sheath, such as the catheter tube 74 seen in FIGS. 6-9. The bendable section 90 comprises a distal segment 92 extending from a first distal ring 94a to a first proximal ring 94b. The bendable section 90 can be used with a variety of delivery systems, including any of the delivery systems described herein or similar delivery systems.

In some implementations, between the proximal and distal rings 94a, 94b is a length of flexible tubing 96. The flexible tubing 96 may be formed by a Nitinol tube having laser-formed circumferential cuts therein to allow flex. The bendable section 90 further comprises a proximal segment 98 extending from a second distal ring 100a to a second proximal ring 100b, with a length of flexible tubing 102 therebetween. As will be seen below, the first proximal ring 94b may be conjoined with the second distal ring 100a into a single intermediate ring 104. A plurality of pull wires 110 are seen extending into the proximal end of the bendable section 90, as will be explained below.

Catheter technology may be limited to one or two planes of flexibility. That is, there may be a first pull wire extending along one side of the delivery system and attach to bend the access sheath in a first direction or plane. In some implementations, there may be a second pull wire extending along the delivery system at a 90° angular rotation from the first pull wire. In some implementations, the second pull wire may be attached to bend the access sheath in a second direction in a plane 90° from the first directional plane. With only one or two planes of flexibility and/or only one or two pull wires, complicated maneuvers may not be possible due to the limited nature of the simple pull wires.

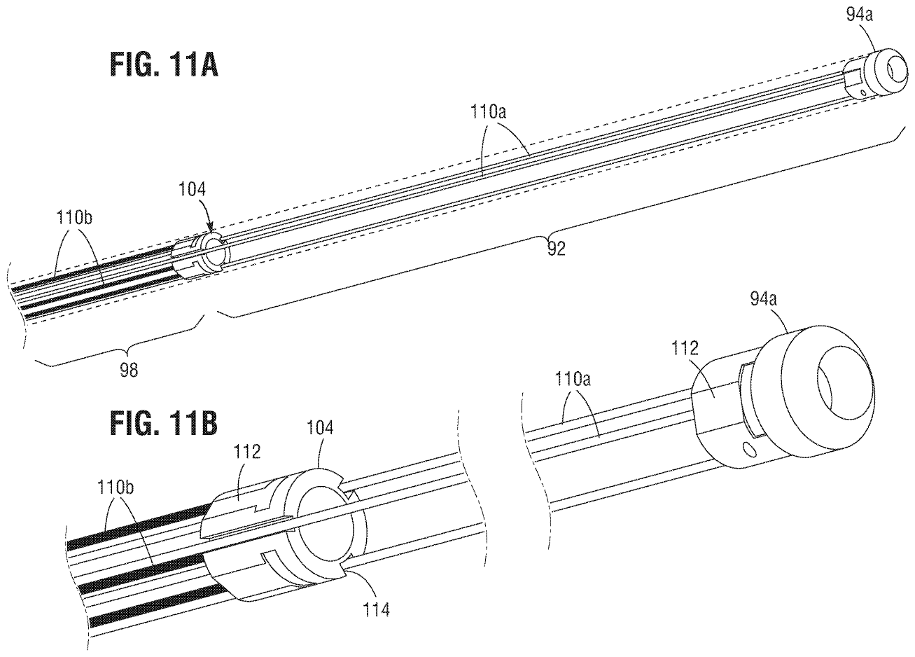

FIGS. 11A and 11B show perspective views of inner pull wires 110a, 110b useable within the bendable section 90 of FIG. 10 for more complicated movements. In some implementations, a first set of pull wires 110a extends along the bendable section 90 to the distal first ring 94a of the distal segment 92. In some implementations, the first set of pull wires 110a are anchored to the first ring 94a. In some implementations, they are anchored to the first ring 94a by, for example, T-shaped grommets 112 that fit within similarly shaped and sized slots in the first ring.

In some implementations, there are three wires in the first set of pull wires 110a angularly spaced 120° apart. In some implementations, each of the first set of pull wires 110a extends from a proximal end of the delivery system and passes through axial channels 114 in the intermediate ring 104 (such as formed by the first proximal ring 94b may be conjoined with the second distal ring 100a). In some implementations, the axial channels 114 are also angularly spaced 120° apart.

In some implementations, in between the axial channels 114, a plurality of T-shaped grommets 112 anchor the second set of pull wires 110b. Supplying six pull wires 110a, 110b that terminate at different locations thus provides innumerable options for bending of the section 90. That is, the distal segment 92 can be bent in one of three first directions, while proximal segment 98 can also be bent in one of three second directions in-between the first three directions. The delivery system can thus be bent in various direction in 60° increments, or first in one direction for the proximal segment 98 and a different direction for the distal segment 92.

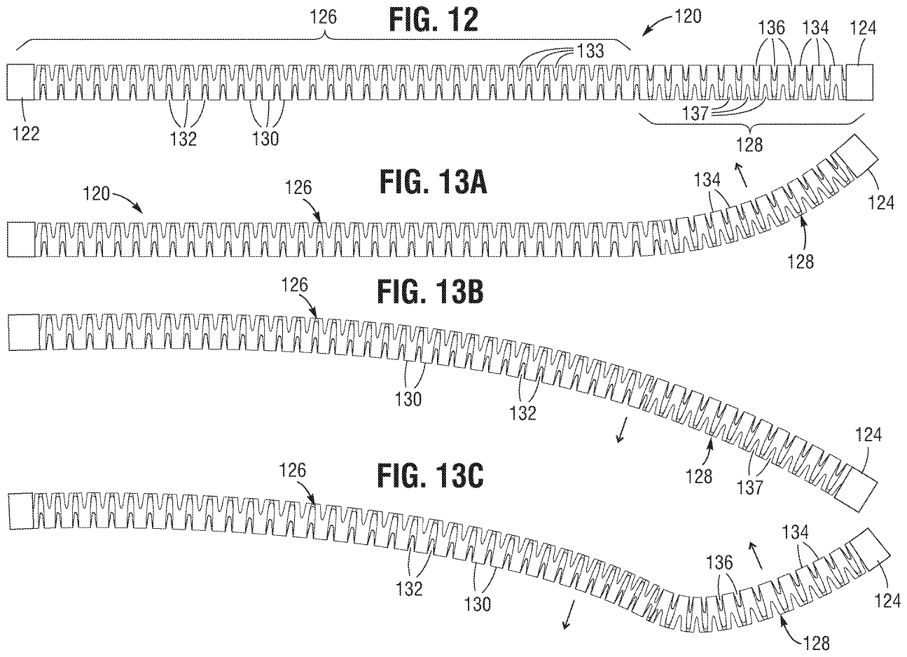

FIG. 12 is an elevational view of an example tubular bendable section 120 of a sheath portion of a delivery system, and FIGS. 13A-13C show different ways the bendable section can flex. The bendable section 120 extends from a proximal ring 122 to a distal ring 124. In between the rings are a first or proximal segment 126 in series with a second or distal segment 128, with the distal segment being shorter than the proximal segment. The bendable section 120 can be used with a variety of delivery systems, including any of the delivery systems described herein or similar delivery systems.

In some implementations, the proximal segment 126 comprises a series of solid ribs 130 that are flexibly connected together for bending and have relatively large gaps 132 therebetween on a lower circumferential side thereof than gaps 133 on an underside. The distal segment 128 also has a series of solid ribs 134 that are flexibly connected together for bending. Relatively larger gaps 136 in the distal segment 128 are located on an upper circumferential side thereof opposite the large gaps 132 in the proximal segment 126, and opposite smaller lower gaps 137. Both the proximal and distal segments 126, 128 are capable of flexing, but due to the relatively larger gaps 132, 136, and distribution of pull wires (not shown) within the tubular bendable section 120, are adapted to bend in opposite directions in only one plane, as will be explained. The solid ribs 130, 134 may be formed of a highly flexible material, such as Nitinol, and be formed by a solid tube with cuts in the illustrated configurations, or may be helically arranged coils with relatively tighter and looser coil spacing.

With reference first to FIG. 13A, a first pull wire (not shown) extends through the proximal ring 122 and is anchored at an upper circumferential side of the distal ring 124. Tension on the first pull wire thus causes upward bending of the distal segment 128, which is facilitated by the relatively larger gaps 136 in the distal segment.

FIG. 13B shows a bending configuration where primarily the proximal segment 126 is caused to flex. A second pull wire (not shown) extends through the proximal ring 122 and is anchored at a lower circumferential side of the distal ring 124. Tension on the second pull wire causes downward bending of the proximal segment 126, facilitated by the relatively larger gaps 132 in the proximal segment. Due to the generally flexible nature of the distal segment 128, it also may bend but to a lesser degree than the proximal segment 126 because the gaps 137 on the lower side are smaller than the gaps 136 on the upper side.

Finally, FIG. 13C shows a double bend where both pull wires top and bottom are in tension. As with FIG. 13B, the proximal segment 126 bends downward due to the larger gaps 132, while the distal segment 128 bends upward because of the larger gaps 136. It should be understood that the relative lengths of the segments 126, 128 may vary as well as the number of segments. That is, there may be three of the bendable segments oriented with their larger gaps at 120° apart, to enable even greater flexibility.

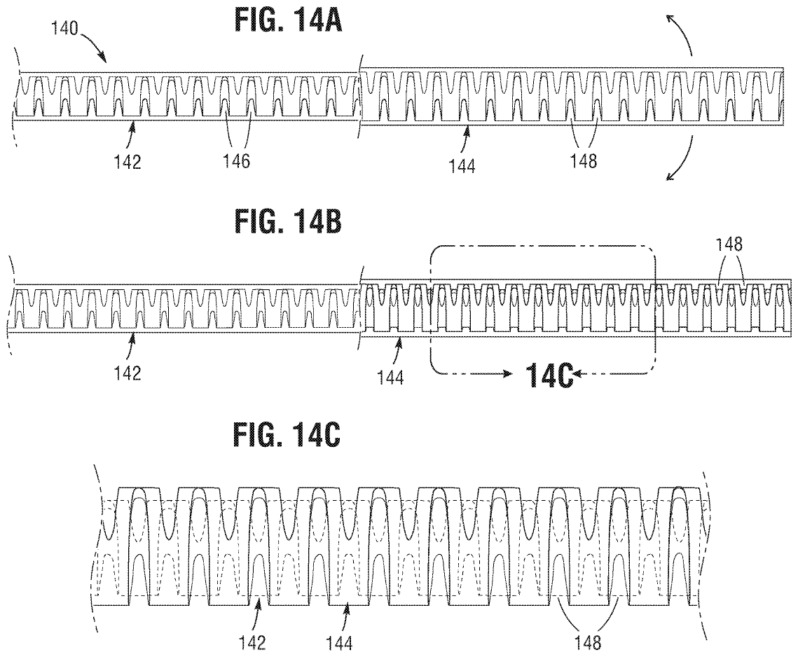

FIG. 14A is a partially cutaway elevational view of a section of a further bendable section 140 of the sheath portion of a valve delivery system having concentric tubes that can be rotated into alignment for bending as shown or rotated out of alignment to resist or inhibit bending. In some implementations, an inner tube 142 may be formed by a series of connected solid ribs having larger gaps 146 on a lower circumferential side thereof. At the same time, a concentric outer tube 144 has larger circumferential gaps 148 on the lower side also. The width and spacing of the gaps 146, 148 are equivalent so that they align in the illustrated position, thus enabling the concentric tubes to be able to bend in one or both directions, as indicated by the arrows. In this situation, both tubes 142, 144 have larger gaps on the lower circumferential side, meaning they can bend further downward than upward. The bendable section 140 can be used with a variety of delivery systems, including any of the delivery systems described herein or similar delivery systems.

FIGS. 14B and 14C show the same concentric tubes 142, 144 having been rotationally misaligned to inhibit bending. Namely, the smaller tube 142 has been rotated so that the larger gaps 146 are now oriented upward, while the larger gaps in the bigger tube 144 remain oriented downward. Because the gaps in both tubes have similar spacing, the solid portions of the ribs on the smaller tube 142 are now aligned with the gaps 148 on the larger tube 144, thus creating rigidity and inhibiting bending.

The ability to rotate concentric tubes and stiffen the overall sheath 140 may be important when advancing the sheath through the sometimes tortuous vasculature. That is, flexibility may be needed in one location, while stiffness is needed in another. Various arrangements, designs, and/or patterns may be used on the concentric tubes to accomplish and/or optimize this.

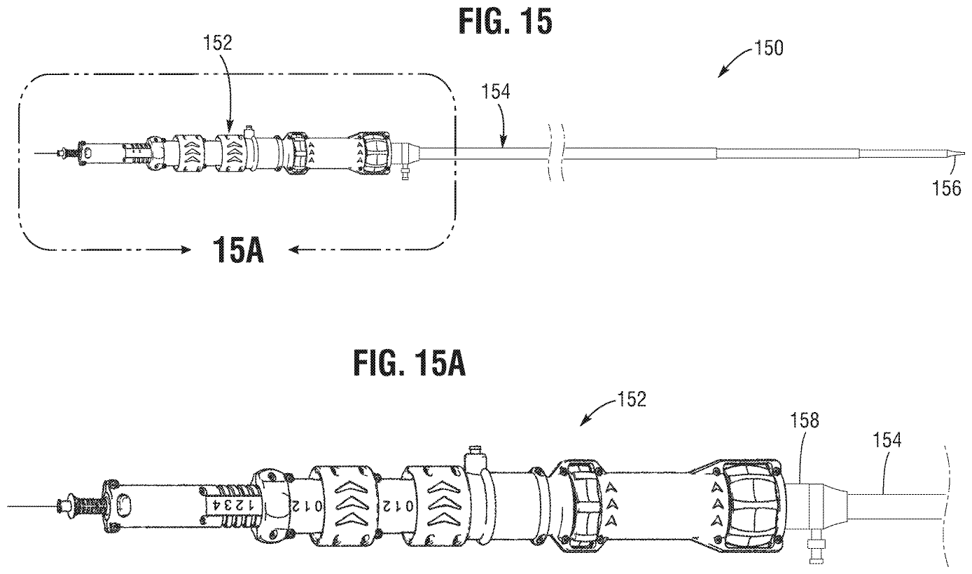

FIG. 15 is an elevational view of an example delivery system 150 (e.g., illustrated as a detachable transcatheter delivery system, but can be any of a device delivery system, treatment device delivery system, repair device delivery system, replacement device delivery system, implant delivery system, prosthetic valve delivery system, etc.), and FIG. 15A is an enlargement of a proximal control handle 152 thereof.

In some implementations, as will be explained, a sheath assembly extending from a proximal sheath 154 to distal end. An optional distal nose cone 156 can be at the distal end (though a nose cone is not required). In some implementations, the delivery system or sheath assembly may house a device, such as a prosthetic valve or other treatment device, therein (not shown).

In some implementations, the sheath assembly can be configured to be detachable from the control handle 152. By making the sheath assembly detachable from the control handle 152, the cost of each delivery system can be greatly reduced in addition to reducing the amount of waste provided from one time use of plastic components in the handle. In some implementations, the sheath assembly is a single-use component made of relatively inexpensive plastics, while the control handle 152 can be manufactured of stainless steel or other more durable and dimensionally precise materials. Higher-quality components for the proximal control handle 152 benefits the physician in terms of an enhanced tactile feel and feedback, and potentially increases precision movements as the handle components are designed for multiple uses.

The enlarged view of FIG. 15A shows the proximal sheath 154 coupled to the handle 152 via a junction hub 158. In some implementations, as will be explained below, the junction hub 158 provides a bayonet-style connector such that the sheath assembly can be easily coupled to and decoupled from the handle 152. First, however, it is instructive to understand the various movable components within the distal sheath assembly.

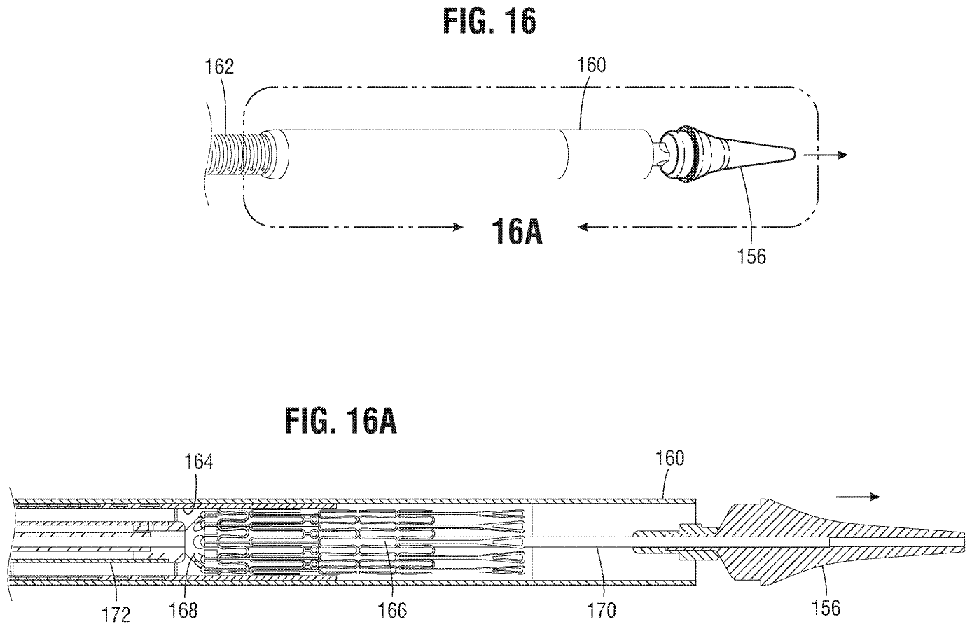

FIG. 16 is a perspective view of a distal end of the delivery system of FIG. 15 following distal displacement of the nose cone 156, and FIG. 16A is a sectional view thereof. An outer sheath or capsule 160 provides a protective cover around a device (illustrated as a transcatheter heart valve or prosthetic heart valve by way of example, but other devices are possible, e.g., repair devices, other treatment devices, etc.) and abuts the nose cone 156 (though not every delivery system includes a nose cone) during advancement through the vasculature. A bendable sheath 162 is provided just proximal to the capsule 160.

With reference to FIG. 16A, an intermediate sheath 164 at the end of the bendable section 162 surrounds and constrains a proximal end of the device or prosthetic heart valve 166 (while shown as a heart valve by way of example, other devices are possible).

Although not shown in great detail, proximal struts on the device or heart valve 166 are captured within apertures in a conical retention member 168 provided at the end of an inner catheter 172. A small diameter catheter or tube 170 extends through the inner catheter 172 and through the device or heart valve 166 to be coupled with the nose cone 156. Although not shown, a guidewire typically extends through the tube 170 to facilitate advancement through the vasculature.

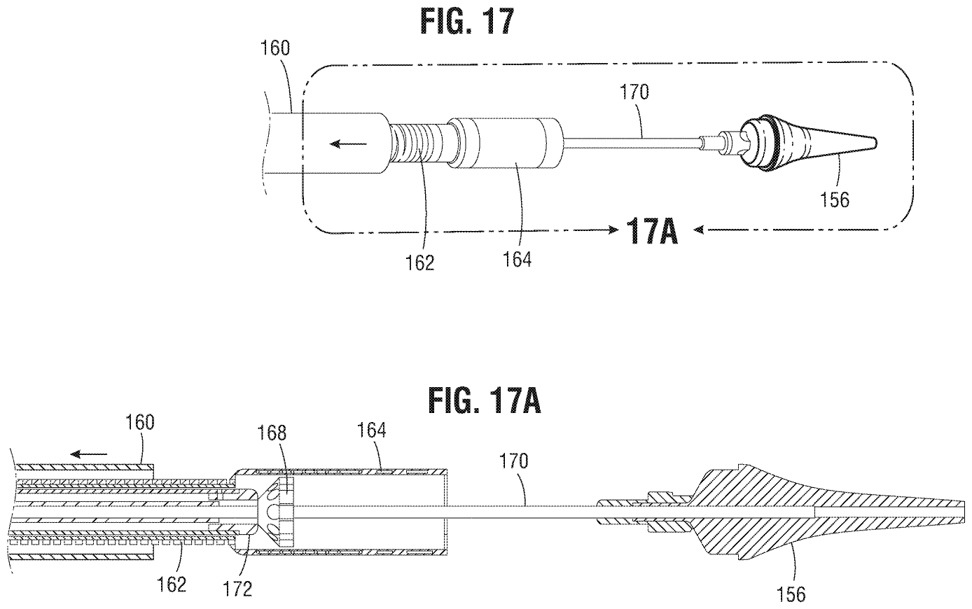

FIG. 17 illustrates the distal end of the delivery system following proximal displacement of the outer capsule 160 in order to expose the device or prosthetic heart valve 166 (not shown for clarity), and FIG. 17A is a sectional view thereof. The proximal end of the device/heart valve 166 remains constrained by the retention member 168 and the intermediate sheath 164. At this point, adjustments to the position of the heart valve can be done under visualization, such as fluoroscopy.

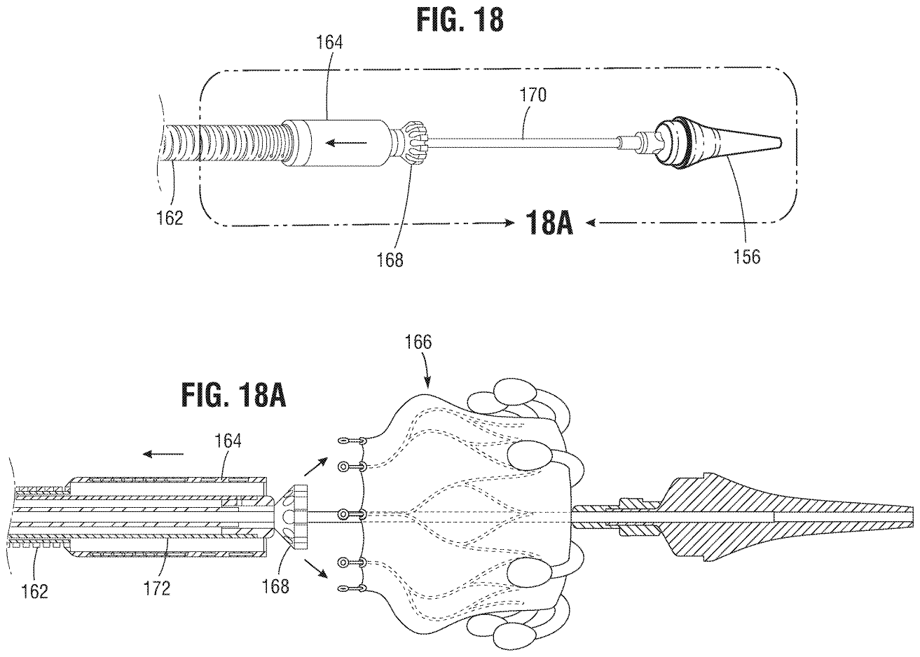

FIG. 18 shows the distal end of the delivery system following proximal displacement of the intermediate sheath 164 to expose and allow expansion of the device or prosthetic heart valve 166, and FIG. 18A is a sectional view thereof. The proximal struts on the device/heart valve 166 spring outward when the intermediate sheath 164 retracts back from around the retention member 168 to fully expand the valve. At this stage, the device 166 is implanted and the delivery system can be retracted and removed from the body. If the device 166 is tethered via sutures or the like to the delivery system, as with some systems, it may be constricted back into the sheath 164 and repositioned.

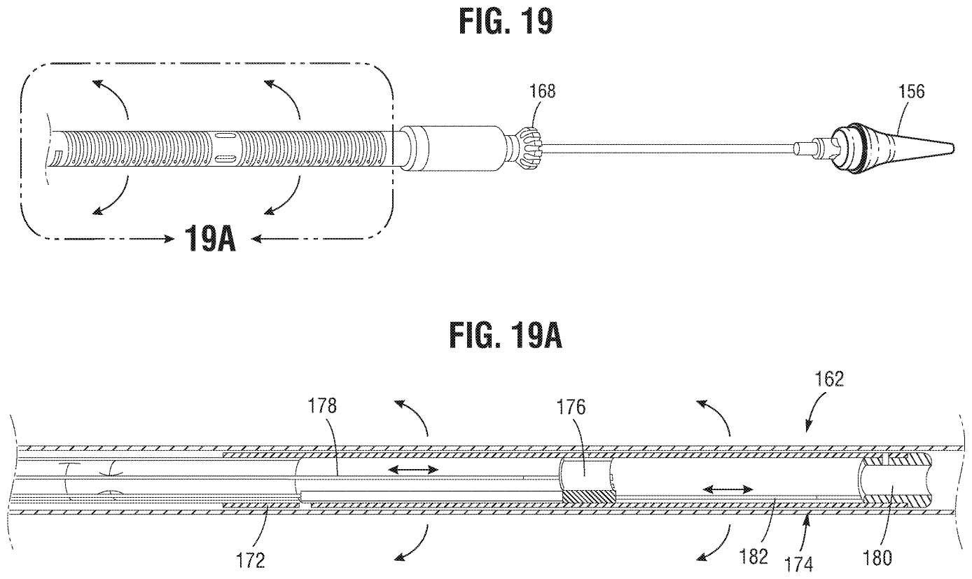

FIG. 19 shows the bending capacity of the bendable section 162. Bending is typically accomplished prior to expanding the device or prosthetic heart valve 166, but may also be useful if the valve is re-constricted for repositioning. Again, the device 166 is not shown, but would otherwise be positioned between the retention member 168 and the nose cone 156.

FIG. 19A is a sectional view of the bendable section 162 which has a tubular inner section 172 in which a first ring 176 is fastened, both within an outer flexible sheath 174. A pull wire 178 connects on one side of the first ring 176 and can be moved axially back and forth. Tension on the pull wire 178 thus bends the section 162 in that direction, and release of tension allows the section to return to a straightened orientation.

Similarly, in some implementations, a second ring 180 spaced distally from the first ring 176 fastens to an end of the inner section 172 and has attached on one side a second pull wire 182. Tension on the second pull wire 182 bends the section 162 in that direction, and release of tension allows the assembly to straighten again.

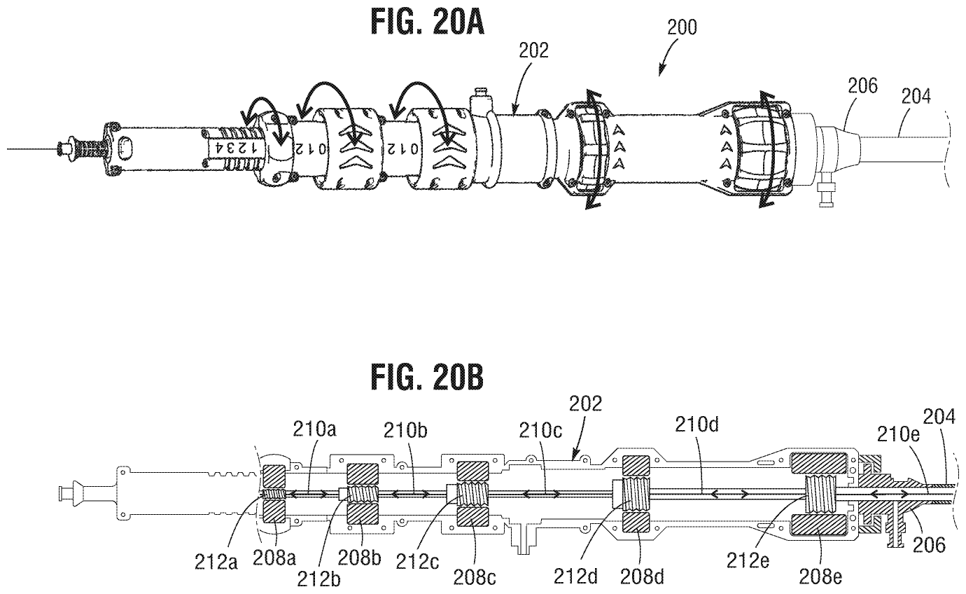

Now with reference to FIG. 20A, an elevational view of an example delivery system 200 and control handle 202 of the present application indicates a number of control dials thereon capable of rotation. The control dials separately displace the various concentric tubes and pull wires previously discussed. One, some, or all of the control dials described and illustrated here can be used with any of the delivery systems described herein or similar delivery systems.

FIG. 20B is a partial sectional view illustrating an example engagement between the detachable sheath assembly and the control handle 202. The detachable sheath assembly has an outer sheath 204 and connecting hub 206. The connecting hub 206 engages a distal end of the handle 202, as will be explained below. A number of concentric tubes project from the connecting hub 206 in a proximal direction into an interior cavity within the handle 202. The handle 202 incorporates a plurality of rotating annular members 208a, 208b, 208c, 208d, 208e which are rotated by the exterior dials.

In some implementations, concentric tubes extending from the detachable sheath assembly include a small inner tube 210a which projects farthest to the left within the handle 202 and has a carriage 212a with outer threading thereon. In some implementations, the annular member 208a engages the threaded carriage 212a, which can be displaced axially by rotating the annular member.

In some implementations, a first group of one or more pull wires 210b terminates at a threaded carriage 212b, while a second group of one or more pull wires 210c terminates at a threaded carriage 212c. In some implementations, the annular members 208b and 208c engage and displace, respectively, the threaded carriages 212b and 212c. In some implementations, the annular member 208d engages a threaded carriage 212d at the proximal end of an intermediate tube 210d. In some implementations, rotation of the annular member 208d axially displaces the carriage 212d and tube 210d. Finally, in some implementations, the annular member 208e engages a threaded carriage 212e at the proximal end of larger tube 210e, which can be axially displaced thereby.

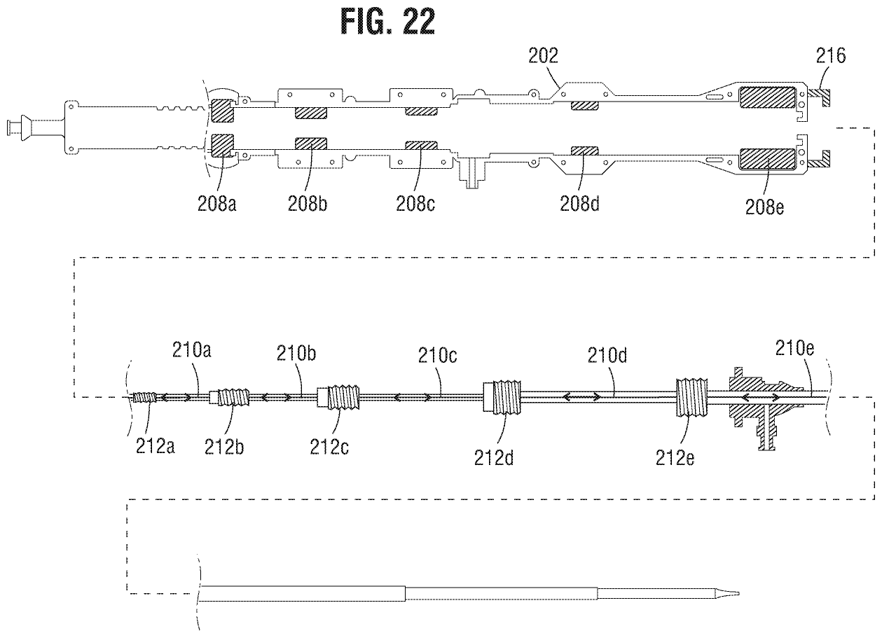

FIG. 22 is an exploded view of the detachable delivery system and handle 202 of FIG. 20A. A single wire is as shown for pull wires 210b and 210c, though there may optionally be more than one.

It should be understood that axial displacement of the carriages 212a-e thus controls movement of the various concentric tubes and pull wires within the delivery system, as explained above. Engagement between the carriages 212a-e and the annular members 208a-e may be done in a number of ways. For example, each of the annular members 208a-e may be split halves that can be retracted outward and then displaced inward to engage the threaded on the carriages 212a-e. Alternatively, the entire assembly of the delivery system that extends into the control handle 202 can slide axially therein until turn and locked in place, as explained below. Different numbers of control knobs and corresponding connections and components can be used for various delivery systems.

Those of skill in the art will understand there are a number of ways of coupling the annular members to the carriages to facilitate driving motion of the various components by turning the exterior dials on the handle 202. For instance, the exterior dials may be readily engaged with the annular members 208a-e to displace them, which in turn engage and axially displace the carriages 212a-e which do not need to be threaded.

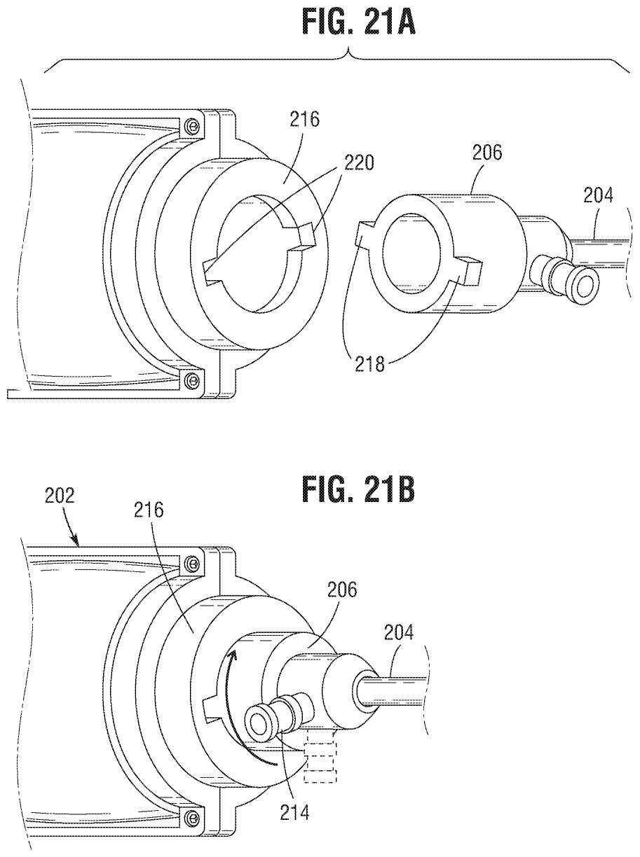

FIG. 21A is an exploded perspective view of a distal end of the example control handle 202 of FIG. 20A adjacent a proximal end of the detachable sheath assembly. FIG. 21B shows the two components being coupled together using a bayonet-type lock (other lock and connection types are also possible). In some implementations, the connecting hub 206 has a pair of outwardly projecting lugs 218 which are sized and shaped to pass through opposite notches 220 in a hollow cylindrical housing 216 on the handle. By inserting the lugs 218 through the notches 220 and rotating clockwise by 90°, for example, the delivery system and its concentric tubes can be locked in place within the control handle 202. As mentioned above, this locking rotation may cause engagement between the annular members 208a-e within a handle 202 and the carriages 212a-e of the delivery system. Of course, other ways to couple the delivery system to the handle 202 are contemplated.

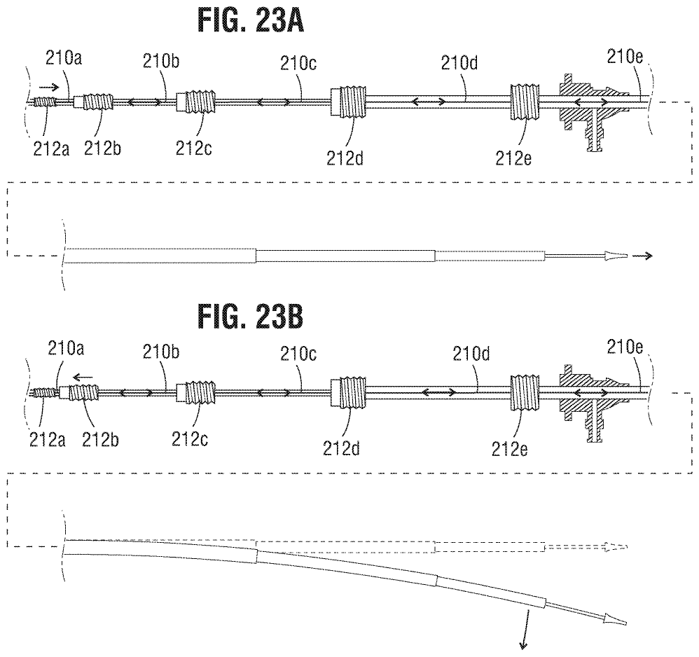

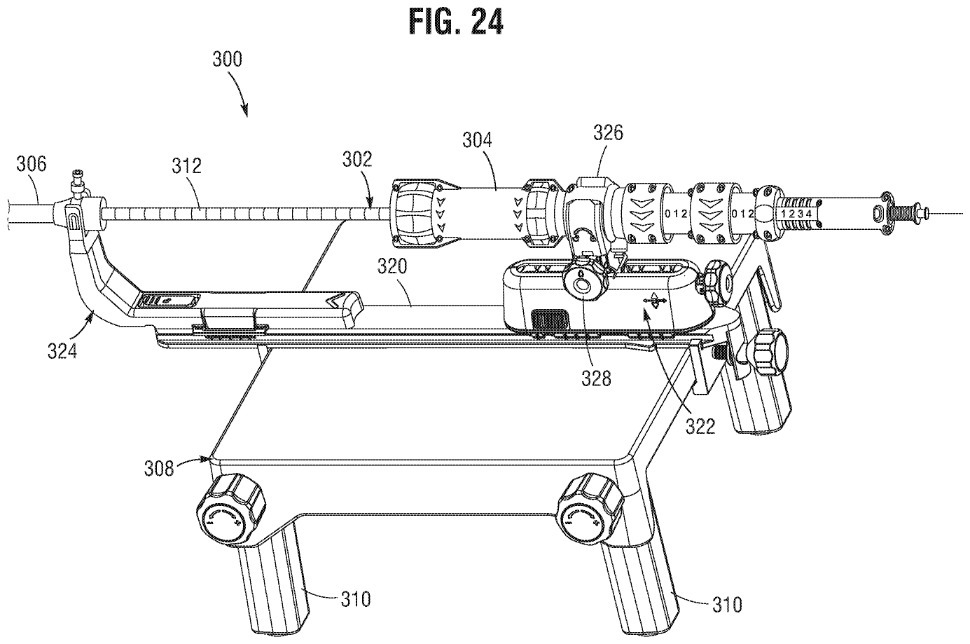

FIGS. 23A and 23B are isolated views of that portion of the detachable delivery system of FIG. 20A that projects within a handle 202, as well as a distalmost end of the sheath assembly showing two possible modes of operation thereof. In FIG. 23A, the smallest carriage 212a as being displaced to the right which ultimately pushes the nose cone 156 to the right. In FIG. 23B, the carriage 212b and first pull wire 210b are displaced to the left, which causes bending of the distal sheaths as shown.

FIG. 24 is a perspective view of an assembly 300 of a proximal end of an example delivery system 302 with a control handle 304 and access hub 306 held in a stabilization system or support mount 308. In some implementations, the stabilizer or support mount 308 has four legs 310 for mounting above a table surface within the operating theater, and adjacent the subject's upper leg. Providing the stabilization system or support mount 308 helps control the position and angle of the delivery system 302 in the process of and during introduction an outer access sheath 312 into the subject's body, typically through the access hub 306 and its sheath through an incision in the upper leg leading to the femoral vein. Of course, the same support mount 308 can be used for other access pathways.

In some implementations, the stabilization system or support mount 308 has a linear track 320. In some implementations, a proximal clamp assembly 322 and/or a distal clamp assembly 324 are mounted on the linear track 320. In some implementations, both clamp assemblies 322, 324 can be displaced along the track 320 to customize the relative spacing between the control handle 304 and access hub 306, as well as to modify their relative heights and/or angles in one such adjustable support mount 308 is disclosed in U.S. Patent Publication No. 2020/0108225 assigned to Edwards Lifesciences Corporation of Irvine, CA, the disclosure of which is expressly incorporated herein in its entirety for all purposes.

In some implementations, the proximal clamp assembly 322 features a clamp 326 having jaws that closely surround a section of the control handle 304 and are alternately tightened and loosened using a control knob 328.

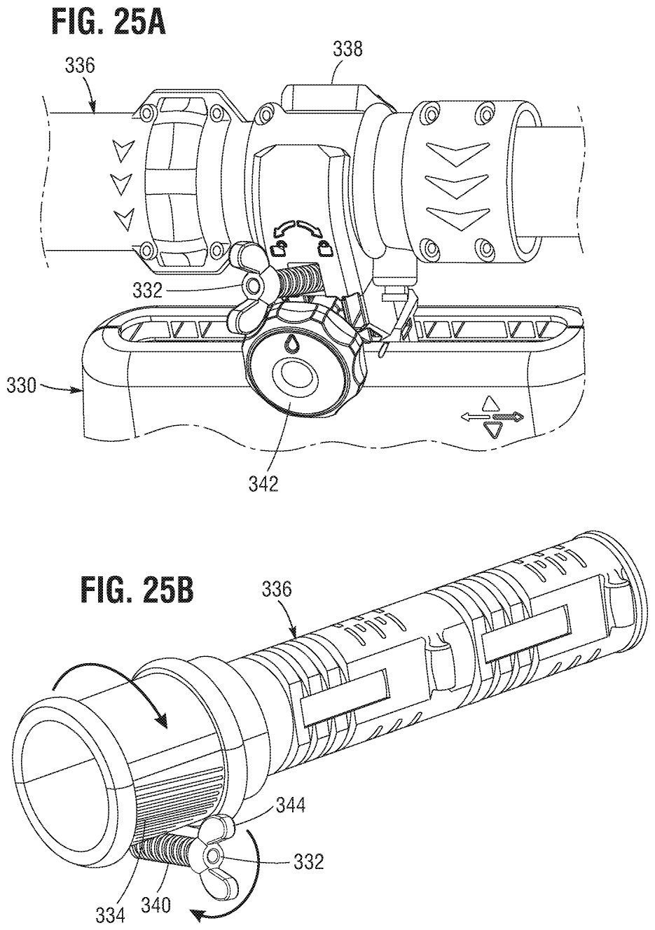

FIG. 25A is a partial perspective view of a clamp region of a support mount or clamp assembly 330 of the present application showing a rotational adjustment bolt 332, and FIG. 25B is a perspective view with only a portion of the delivery system shown with which the rotational adjustment bolt interacts. In some implementations, as shown in FIG. 24, gross rotational adjustment of the delivery system is accomplished by loosening the clamp 326 and physically rotating the control handle 304, which can be rotationally fixed with respect to the distally-extending outer sheath 312. This adjustment requires time, and the somewhat awkward process of loosening the clamp 326, rotating the handle 304 a specific sometimes minimal amount, and then re-tightening the clamp 326 around the handle. The support mount 330 of FIG. 25A provides one solution.

FIG. 25B illustrates a threaded or ribbed portion 334 of the control handle 336 that is within the jaws of the clamp 338 and interacts with the rotational adjustment bolt 332. In some implementations, the outer threads 340 on the bolt 332 act like a worm screw on the ribbed portion 334 to cause rotation of the control handle 336 about its axis, as shown. In some implementations, the user need only loosen the control knob 342 that opens and closes the clamp 338, and then turn the rotational adjustment bolt 332 for fine rotational positioning. In some implementations, the bolt 332 has a wingnut-type outer end 344, but the outer end could also the knurled or otherwise ergonomically shaped like the control knob 340 for ease of manual rotation.

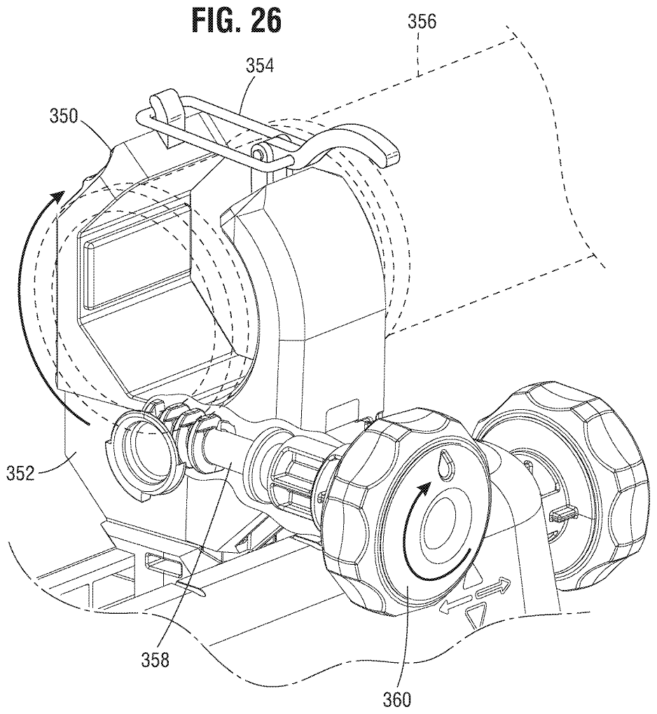

FIG. 26 is a perspective view of an example clamp of a stabilization system or support mount indicating another configuration for rotational adjustment of the delivery system. In some implementations, the jaws 350 of the clamp 352 are closed at an upper end using a toggle-like latch 354. The control handle 356 is shown in phantom within the jaws. In some implementations, the diameter of the concave jaws 350 when closed is approximately the same as or slightly larger than the section of the control handle 356 that is grasped. More particularly, closing the jaws 350 provides positional stability without creating a tight interference fit.

In some implementations, a lower generatrix of the section of the control handle 356 within the jaws 350 is threaded or otherwise ribbed as shown at 334 in FIG. 25B, and cooperates with worm-screw threading on a rotational adjustment bolt 358. In some implementations, the bolt 358 may be controlled by an outer control knob 360, and extends perpendicularly underneath the control handle 356. Although not shown, the rotational adjustment bolt 358 is journaled for rotation in a stationary position such that the worm-screw threads act on the threaded lower generatrix of the control handle 356 for fine rotational adjustment thereto. In some implementations, there is no need to even loosen the clamp on the control handle, it is held firm positionally and simply rotated incrementally about its own axis using the control knob 360.

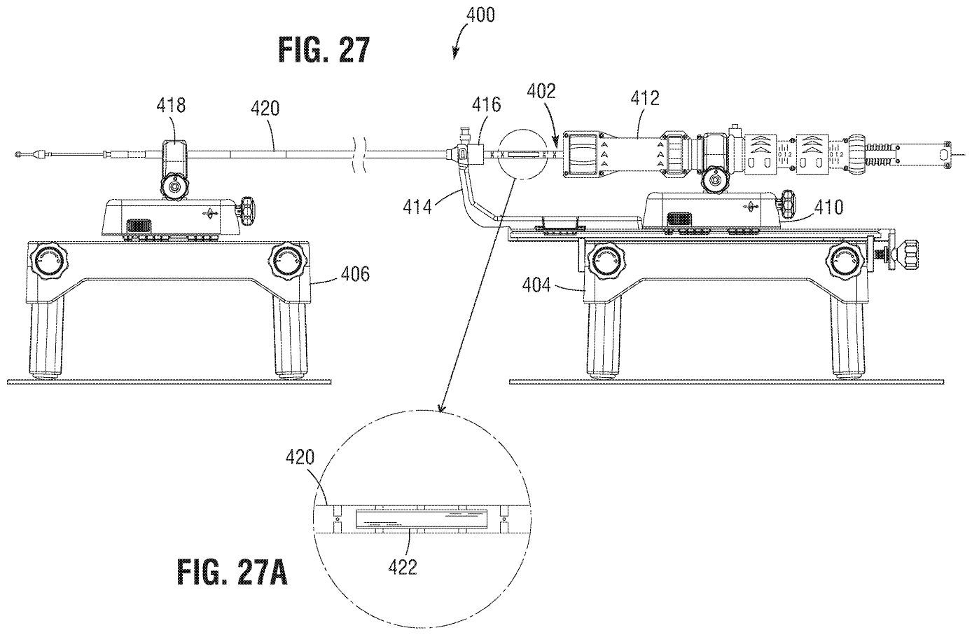

FIG. 27 is a side elevational view of an assembly 400 of a delivery system 402 with a proximal end held on a primary support mount 404, as described above. In some implementations, the assembly 400 includes a secondary support mount 406 that holds a distal end of the delivery system 402, which can help enable one-handed device (e.g., valve, etc.) loading. In some implementations, as before, the primary stabilization system or support mount 404 has a proximal clamp assembly 410 holding a control handle 412, and a distal clamp assembly 414 that holds an access hub 416.

In some implementations, a secondary stabilization system or support mount 406 is provided that has a clamp assembly 418 that supports a distal portion of an outer sheath 420 of the delivery system 402. The benefit of adding the secondary support mount 406 will be described below. FIG. 27A is an enlargement of a proximal portion of the outer sheath 420 closely adjacent the control handle 412 and illustrating a positioning window 422 therein, whose purpose will also be described below.

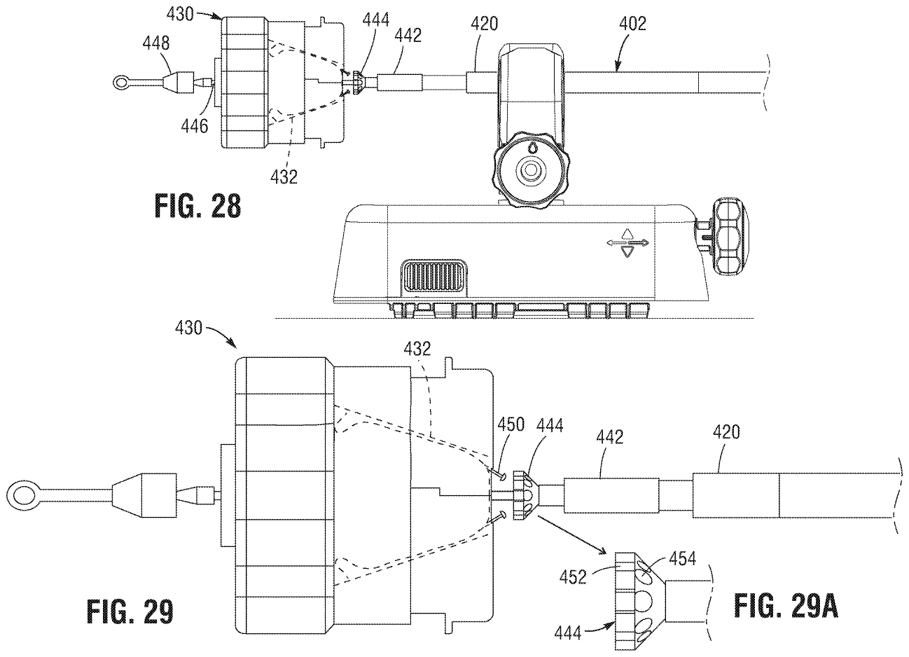

FIG. 28 is a side elevational view of a distal end of the assembly 400, and FIG. 29 is an enlarged view of a distal end of the delivery system 402 showing a heart valve crimping assembly 430. In some implementations, the crimping assembly 430 comprises a cylindrical apparatus that contains the device or prosthetic heart valve 432 (while depicted for example as a prosthetic heart valve other devices can be used with the assembly 400 and delivery system 402) in an initially expanded configuration, and through manual manipulation of relatively movable elements of the assembly 430 the device/heart valve is crimped and loaded onto the delivery system 402. Crimping assemblies 430 can include features and/or components disclosed in U.S. Patent Publication Nos. 2017/0367821 and/or 2022/0346993, assigned to Edwards Lifesciences and expressly incorporated by reference herein in their entireties for all purposes.

In some implementations, the crimping assembly 430 and device/heart valve 432 can be positioned on the distal end of the delivery system 402 in the operating theater, or the pre-assembled with the delivery system 402 at the manufacturing stage. In some implementations, the device or heart valve 432 is stored dry and packaged along with the crimping assembly 430 and delivery system 402. In some implementations, the crimping assembly 430 can include two separable halves to facilitate assembly and removal around the delivery system 402.

In some implementations, the delivery system 402 has the large outer sheath 420 through which one or more concentric inner tubes or catheters pass. In some implementations, an intermediate tube 442 extends from the distal end of the outer sheath 420 terminating in a conical valve retention member 444, much like the retention member 168 described above in earlier implementations. An inner tube 446 passes through the intermediate tube 442 and continues (to the left) to a distal nose cone 448 on the distal side of the crimping assembly 430.

In some implementations, once the prosthetic heart valve 432 has been crimped down in size and loaded into the delivery system 402, an optional nose cone 448 provides a cap on the distal end of the outer sheath 420. In some implementations, the tapered shape of the nose cone 44 and facilitates passage of the delivery system 402 through the vasculature to the target valve implantation site. The nose cone 448 is optional and need not be included. The nose cone 448 can optionally include one or more of the arrays and/or other features described with respect to FIGS. 4-5.

In some implementations, with particular reference to FIG. 29, the device or prosthetic heart valve 432 is shown in dashed line within the crimping assembly 430 in a partially crimped state. In some implementations, as a user manually manipulates the crimping assembly 430, the device/heart valve is gradually urged to the right through a conical passage which reduces its radial size. Expandable and flexible struts on the heart valve terminate at a proximal end in free ends 450 having enlarged beaded ends for capture by the valve retention member 444.

In some implementations, as best seen in the enlargement of FIG. 29A, the retention member 444 has a series of axial slots 452 opening to its distal end through which the free ends 450 pass such that their enlarged beaded ends can be captured within recesses 454 opening to the proximal side of the retention member. Once the free ends 450 are distributed around the retention member 444, a larger outer sheath may be advanced around the retention member to prevent the free ends from springing outward again.

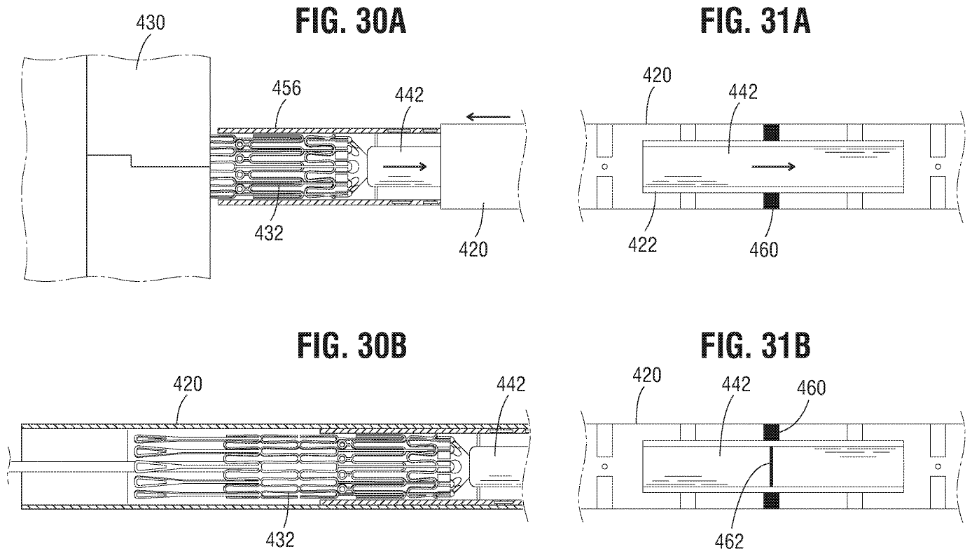

FIGS. 30A and 30B are sectional views through the distal end of the delivery system 402 showing crimping and loading of the device or heart valve 432 into the access sheath. As mentioned above, the proximal free ends 450 of the expandable struts of the heart valve 432 are captured within the recesses 454 of the retention member 444. Either the intermediate tube 442 is retracted proximally or a larger secondary sheath 456 is advanced distally, or both, to constrain the free ends 450 from coming loose. At this stage, the remainder of the crimped device 432 is retracted within the delivery system 402 by distally advancing the outer sheath 420 or proximally retracting the intermediate tube 442, as indicated in FIG. 30A. The device/heart valve 432 needs to be retracted within the delivery system 402 to a particular location near the distal end thereof for best results when advancing in delivering the device/heart valve to the target native valve.

Some procedures for crimping, loading and properly positioning heart valves in delivery systems may require two people. In some implementations, a first technician manipulates the relatively movable tubes using the control handle 412, seen in FIG. 27. In some implementations, the first technician advances the intermediate tube 442 until it contacts the crimping assembly 430. At the same time, a second technician actuates the crimping assembly 430 to both crimp the heart valve 432 and displace it proximally out of the crimping assembly toward the delivery system 402. Positioning the device/heart valve 432 involves the first technician again manipulating the delivery system tubes using the control handle 412 until the second technician observing the position of the heart valve 432 from the distal end of the assembly 400 says to stop. By requiring two technicians and relying on the second technician's powers of observation, the procedure is labor-intensive among other challenges.

To improve this process of retracting the device/heart valve 432 into the delivery system 402, a positioning window 422 in the outer sheath 420 can be provided. FIGS. 31A and 31B are side elevational views of the positioning window on the proximal portion of the outer sheath during the steps illustrated in FIGS. 30A and 30B, respectively.

In some implementations, a length of the intermediate tube 442 which moves with the heart valve 432 is exposed through the positioning window 422, which may be open or simply be a transparent section of the outer sheath 420. In some implementations, the outer sheath 420 has a relatively large thick circumferential hash mark 460 visible on its exterior surface at the midpoint of the positioning window 422. As the intermediate tube 442 moves proximally relative to the outer sheath 420, a circumferential fiducial marking 462 on the intermediate tube eventually comes into view through the positioning window 422. A technician manipulating the control handle 412 can easily see when the fiducial marking 462 lines up with the hash mark 460 on the outer sheath 420, which signals that the heart valve 432 is properly positioned within the delivery system 402. This can all be done by one technician operating the control handle 412, while all along the entire delivery system is mounted securely by the primary and secondary support mounts 404, 406, seen in FIG. 27.

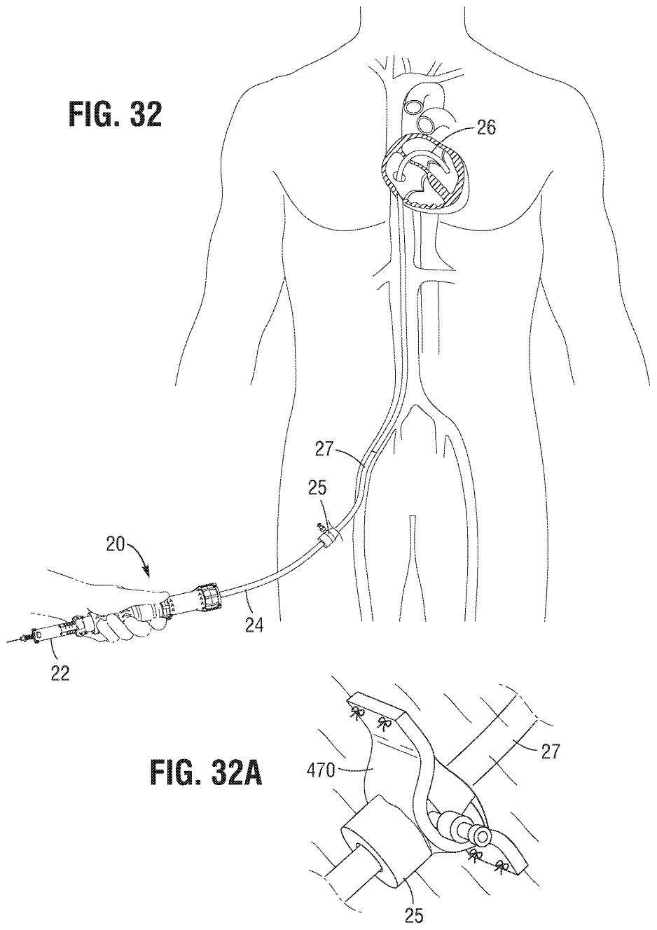

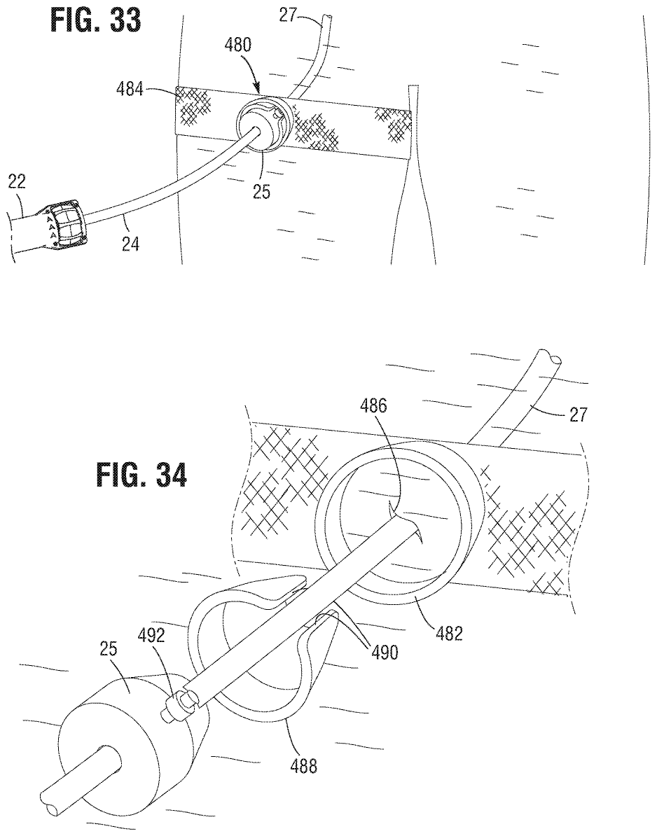

FIG. 32 is a schematic representation of a transcatheter procedure showing advancement of a delivery system 20 through a blood vessel in the leg such as the femoral vein up to the vicinity of the mitral valve within the heart, as in FIG. 1. As stated above, the delivery system 20 has a proximal handle 22 from which an elongated outer sheath 24 extends distally. The outer sheath 24 corresponds to the outer sheath 420 described above in reference to FIGS. 27-32. In some implementations, the outer sheath 24 enters the femoral vein through an access hub 25 housing a series of elastomeric valves along an internal passage to prevent blood leakage around the sheath 24. In some implementations, the access hub 25, in turn, has an access sheath 27 extending a short distance distally therefrom which enters the femoral vein and provides an entrance ramp, so to speak, for the outer sheath 24 of the delivery system 20.

FIG. 32A is an enlargement of an example method for securing the access hub 25 at the incision using sutures. That is, it is preferable to stabilize the access hub 25 immediately adjacent and just outside of the incision to provide a fixed reference point from which to enter the body. One way to accomplish that is to suture a fabric or otherwise flexible strip of material 470 around the hub 25 as shown. In some implementations, the strip of material 470 is split or otherwise has a through bore so as to surround and harness the hub 25, and stitches are used through the ends of the strip into the skin. Securing the hub 25 in this method can be time-consuming among other challenges.

FIG. 33 is a perspective view of an upper leg of a subject (e.g., living subject, simulation, etc.) illustrating a strap-mounted hub docking station 480, and FIG. 33A is an exploded enlargement thereof. The docking station 480 includes a generally tubular dock 482 having a flexible, typically stretchy strap 484 attached thereto and extending in both directions therefrom. The strap 484 is configured to wrap around a portion of a subject's body (e.g., a limb, arm, leg, etc.). In some implementations, the strap is configured to wrap around an upper portion of a subject's leg. In some implementations, the strap is configured to be secured using Velcro, adhesive, tape, or another securing means.

As seen in FIG. 33A, the tubular dock 482 can have a diameter large enough to circumscribe an area surrounding the incision 486. The access hub 25 is then stabilized within the dock 482 using an intermediate sleeve or jacket 488. The access hub 25 can have a tapered distal end, and the jacket 488 may have a tapered shape that closely matches the taper. By providing a longitudinal break or slit 490 on one side of the otherwise annular jacket 488, it may be easily slipped around the access sheath 27 and press fit onto the access hub 25. That is, the slit 490 enables the jacket 488 to flex apart, which can be used to create an interference fit around the access hub 25. The slit 490 also accommodates a side port 492 which is often provided on the access hub 25 for the purpose of aspiration or irrigation.

Subsequently, the access hub 25 having the conical jacket 488 positioned around it can be press fit into the similarly-shaped and sized dock 482. More particularly, the dock 482 may have a conical inner wall which the jacket 488 closely conforms to. The taper on these cooperating parts may be such that they can be simply pressed together to create an interference fit, which holds and stabilizes the access hub 25 at least during the time of the operation. Moreover, the dock 482 may be sized slightly smaller so that it closely receives the access hub 25 itself, thus eliminating the need for the outer jacket 488. However, the outer jacket 488 may be formed of a compressible material which facilitates the interference fit with both the access hub 25 and the dock 482. Similarly, the outer jacket 488 may comprise adhesive on both inner and outer surfaces with a peel-away covering to facilitate temporary attachment to the hub 25 and dock 482.

In some implementations, the outer jacket 488 may be made of a ferromagnetic material such as steel, with the tubular dock 482 being magnetized. For example, the tubular dock 42 may comprise or have mounted thereon a permanent magnet which attracts the steel outer jacket 488. In some implementations, the dock 42 may incorporate an electromagnet which can be switched on or off. Because the jacket 488 is flexible and has the longitudinal slit 480, it easily creates an interference fit around the access hub 25, which is typically plastic.

While the foregoing is a complete description of the preferred implementations of the invention, various alternatives, modifications, and equivalents may be used. Moreover, it will be obvious that certain other modifications may be practiced within the scope of the appended claims.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

What is claimed is:

1. A system, comprising:
a flexible access sheath for advancement of an implant to an implantation site including:
an outer tube defining a first set of gaps; and
an inner tube defining a second set of gaps; and
a pull wire that extends through and that is secured to the flexible access sheath;
a proximal control handle that:
is attached to the flexible access sheath;
is attached to the pull wire;
is configured to tension the pull wire to bend the flexible access sheath; and
includes an actuator for rotating one of the inner tube and the outer tube relative to the other to:
increase a flexibility of the flexible access sheath by bringing into rotational alignment the first and second set of gaps to bend the flexible access sheath toward the first and second set of gaps; and
increase a rigidity of the flexible access sheath by bringing into a rotational misalignment the first and second set of gaps to inhibit bending of the flexible access sheath.

2. The system according to claim 1, further comprising the implant.

3. The system according to claim 2, wherein the implant is a prosthetic heart valve.

4. The system according to claim 3, wherein the inner and outer tubes are concentric.

5. The system according to claim 1, wherein a width and a spacing of individual gaps of the first and second set of gaps are equal such that the first and second set of gaps are longitudinally aligned.

6. The system according to claim 1, wherein the outer tube is rotatable relative to the inner tube.

7. The system according to claim 1, wherein the inner tube is rotatable relative to the outer tube.

8. The system according to claim 7, wherein the inner tube includes a first set of ribs directly opposing the first set of gaps.

9. The system according to claim 8, wherein rotating the inner tube relative to the outer tube aligns the first set of ribs with the second set of gaps such that the ribs inhibit the flexible access sheath from contracting spaces between individual gaps of the second set of gaps.

10. The system according to claim 1, wherein the first and second set of gaps are laser cut into a side of the outer and inner tubes respectively.

11. The system according to claim 1, wherein the outer tube further defines a third set of gaps, and the inner tube further defines a fourth set of gaps.

12. The system according to claim 11, wherein:
the first set of gaps are defined on a first circumferential side of the outer tube and the third set of gaps are defined on a second circumferential side, opposite the first circumferential side, of the outer tube; and
the second set of gaps are defined on a first circumferential side of the inner tube and the fourth set of gaps are defined on a second circumferential side, opposite the first circumferential side, of the inner tube.

13. The system according to claim 12, wherein individual gaps of the first set of gaps alternate with individual gaps of the third set of gaps, and individual gaps of the second set of gaps alternate with individual gaps of the fourth set of gaps.

14. The system according to claim 12, wherein individual gaps of the first set of gaps are larger than individual gaps of the third set of gaps and individual gaps of the second set of gaps are larger than individual gaps of the fourth set of gaps.

15. The system according to claim 14, wherein the flexible access sheath is configured to bend further toward the first circumferential side than the second circumferential side.

16. The system according to claim 12, wherein a central axis of the outer and inner tubes passes through each of the first, second, third, and fourth sets of gaps.

17. The system according to claim 12, wherein the first set of gaps overlaps with the third set of gaps along a longitudinal axis of the outer tube.

18. The system according to claim 12, wherein the flexible access sheath further includes a ring positioned at a distal end of the outer and inner tubes to which the pull wire is secured.

19. The system according to claim 18, wherein the pull wire is a first pull wire which is secured to the ring at the first circumferential side of the inner tube and the control handle further includes a second pull wire secured to the control handle at a first side and to the ring at the second circumferential side at second side.

20. The system according to claim 1, wherein the control handle further includes a control knob to which the pull wire is secured, the control knob configured to tension the pull wire such that the flexible access sheath bends toward the first and second set of gaps.
